SEQUENCE LISTING (7 sequences)

SEQ ID NO: 1 - DNA, 1804 bp, Hordeum vulgare
tccaccgagatgccgacgcacatggcggcgacgatcgattggcgtccatcccgtgcatgctccagtccaccgcaccgccaccaagtgcaaccccctagctagtttaaccagccagagagccgcatccaacttgtgctcgccggcgtacgtgcacacgcgccacccctttacacttgtttattattgcagcttcttcgccccttttggctgcttcttctcccgacatgggctccatcgacatggcggggcttcgcgaaggtacggcgggggagcggcaacgcgtgtcctccctacgtggcggccatgtacgagcaccgccgcgcaacgtgtcccggcgactctcccgtccgtcccgcctataaaggccacccgcgccaatctcctctccacaagcagtcgatccattccaagtgagctaagcaacagcctaaagcgagtccgagtggtgattccagttcgtgtttgtttgagctagatcgtgagacgaagatggcctccaaccagaaccaggggagctaccacgccggcgagaccaaggcccgcaccgaggtgaccgtcgtctccttggtgtctatctatactctgcctgccgcgcgcatgcggcgttgctccggcggtgatctgatatgttcttctgtatctgctgggtgagttgcaggagaagaccgggcagatgatgggcgccaccaagcagaaggcggggcagaccaccgaggccaccaagcagaaggccggcgagacggccgaggccaccaagcagaagaccggcgagacggccgaggccgccaagcagaaggccgccgaggccaaggacaagacggcgcagacggcgcaggcggccaaggacaagacgtacgagacggcgcaggcggccaaggagcgcgccgcccagggcaaggaccagaccggcagcgccctcggcgagaagacggaggcggccaagcagaaggccgccgagacgacggaggcggccaagcagaaggccgccgaggcaaccgaggcggccaagcagaaggcgtccgacacggcgcagtacaccaaggagtccgcggtggccggcaaggacaagaccggcagcgtcctccagcaggccggcgagacggtggtgaacgccgtggtgggcgccaaggacgccgtggcaaacacgctgggcatgggaggggacaacaccagcgccaccaaggacgccaccaccggcgccaccgtcaaggacaccaccaccaccaccaggaatcactagacgcatgcgttcgcgcttaatttccgttcctttagtcgtgtttggtcgttcgagggccttctacatatttcatatttgtatgtttccactctttcatgatttccgctcatttagtgtaagtttgcctccgatttgatgtactcgtctctggttctgtaatgagttataatccatgggctttggtgtaaatggataacgaggacactcgaaggcggcaataaagttgtatgtgatcgaatttctgtattttggtagtgtcaatgaaaacatatattgtgtttcatagatagtgtggcctttaaaatatgcaaatagtctgacccttaaaatatgcaaattagctactgacttcgagacattgtacatgacttaagatgtacactgacttgagacattgtacatgactttaagatgtacactgaagacatggtacatgacgcaaaccaacccattattcctcgatacgttttcaaggaagacatttttttacgatgaatgatatgttgatagaggtatcatatgttcgtagatacgtttttctacgattcttagcaggcatggtac

SEQ ID NO: 2 - protein, 11 aa, Hordeum vulgare
TAQAAKEKAGE

SEQ ID NO: 3 - DNA, 2707 bp, Saccharomyces cerevisiae
ttatgtaatttagttacgcttgactgatgtacatttgagattatcaaaaaaactgcttaagagatggatgatttaattttttagagacgtattaatggaactttttataccttgcccagagcgcctcaagaaaatgatgctgcaagaagaattgaggaaggaactattcatcttacgttgtttgtatcatcccacgatccaaatcatgttacctacgttaggtacgctaggaactaaaaaaagaaaagaaaagtatgcgttatcactcttcgagccaattcttaattgtgtggggtccgcgaaaatttccggataaatcctgtaaactttaacttaaaccccgtgtttagcgaaattttcaacgaagcgcgcaataaggagaaatattatctaaaagcgagagtttaagcgagttgcaagaatctctacggtacagatgcaacttactatagccaaggtctattcgtattactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaactttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatagtataatatgaaaaatatcatttcattggtaagcaagaagaaggctgcctcaaaaaatgaggataaaaacatttctgagtcttcaagagatattgtaaaccaacaggaggttttcaatactgaagattttgaagaagggaaaaaggatagtgcctttgagctagaccacttagagttcaccaccaattcagcccagttaggagattctgacgaagataacgagaatgtgattaatgagatgaacgctactgatgatgcaaatgaagctaacagcgaggaaaaaagcatgactttgaagcaggcgttgctaaaatatccaaaagcagccctgtggtccatattagtgtctactaccctggttatggaaggttatgataccgcactactgagcgcactgtatgccctgccagtttttcagagaaaattcggtactttgaacggggagggttcttacgaaattacttcccaatggcagattggtttaaacatgtgtgtcctttgtggtgagatgattggtttgcaaatcacgacttatatggttgaatttatggggaatcgttatacgatgattacagcacttggtttgttaactgcttatatctttatcctctactactgtaaaagtttagctatgattgctgtgggacaaattctctcagctataccatggggttgtttccaaagtttggctgttacttatgcttcggaagtttgccctttagcattaagatattacatgaccagttactccaacatttgttggttatttggtcaaatcttcgcctctggtattatgaaaaactcacaagagaatttagggaactccgacttgggctataaattgccatttgctttacaatggatttggcctgctcctttaatgatcggtatctttttcgctcctgagtcgccctggtggttggtgagaaaggatagggtcgctgaggcaagaaaatctttaagcagaattttgagtggtaaaggcgccgagaaggacattcaagttgatcttactttaaagcagattgaattgactattgaaaaagaaagacttttagcatctaaatcaggatcattctttaattgtttcaagggagttaatggaagaagaacgagacttgcatgtttaacttgggtagctcaaaatagtagcggtgccgttttacttggttactcgacatatttttttgaaaagaagcaggtaatggccaccgacaaggcgtttactttttctctaattcagtactgtcttgggttagcgggtacactttgctcctgggtaatatctggccgtgttggtagatggacaatactgacctatggtcttgcatttcaaatggtctgcttatttattattggtggaatgggttttggttctggaagcagcgctagtaatggtgccggtggtttattgctggctttatcattcttttacaatgctggtatcggtgcagttgtttactgtatcgttgctgaaattccatcagcggagttgagaactaagactatagtgctggcccgtatttgctacaatctcatggccgttattaacgctatattaacgccctatatgctaaacgtgagcgattggaactggggtgccaaaactggtctatactggggtggtttcacagcagtcactttagcttgggtcatcatcgatctgcctgagacaactggtagaaccttcagtgaaattaatgaacttttcaaccaaggggttcctgccagaaaatttgcatctactgtggttgatccattcggaaagggaaaaactcaacatgattcgctagctgatgagagtatcagtcagtcctcaagcataaaacagcgagaattaaatgcagctgataaatgttaagtaaaagggttgtttttttttttttggaagaaataaggaatccctttgactgctcccaaaaccctcag

SEQ ID NO: 4 - DNA, 1425 bp, Escherichia coli
atgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtag

SEQ ID NO: 5 - DNA, 801 bp, Escherichia coli
gtgacagaaccgttaaccgaaacccctgaactatccgcgaaatatgcctggttttttgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaa

SEQ ID NO: 6 - DNA, 801 bp, Escherichia coli
atgacagaaccgttaaccgaaacccctgaactatccgcgaaatatgcctggttttttgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatgggaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggggaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaa

SEQ ID NO: 7 - DNA, 1425 bp, Escherichia coli
atgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaagggaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacaactttgtgattatgatttgctgggtttccaaacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtag

(19) United States
(12) Patent Application Publication
(10) Pub. No.: US 2005/0186185 A1
(43) Pub. Date: Aug. 25, 2005

(54) Room temperature storage of organs

(76) Inventors: Paul Conrad, Madison, WI (US); Lynn Allen-Hoffmann, Madison, WI (US)

Correspondence Address: J. Mitchell Jones;MEDLEN & CAROLL, LLP, 101 Howard Street, Suite 350, San Francisco, CA, 94105, US

(21) Appl. No.: 10/857,764

(22) Filed: May 28, 2004

Related U.S. Application Data
(60) Provisional application No. 60/474,334, filed on May 30, 2003.

(51) Int. Cl. A61K 48/00; C12N 5/08; A01N 1/02

(52) U.S. Cl. 424/93.21; 435/1.1; 435/371

(57) ABSTRACT

The present invention relates generally to compositions and method for freezing and/or drying organs for storage prior to use. In particular, the present invention relates to the genetic modification of cells used to form organs so that organs formed from the genetically modified cells can be dried. According to the invention, mammalian cells may be modified with genes encoding plant late embryogenesis protein HVA1, trehalose transport protein, or a trehalose synthesis pathway. The invention also provides methods of treating patients with organs that have been preserved by freezing and/or drying.

This application claims priority to provisional patent application Ser. No. 60/474,334 filed May 30, 2003, which is herein incorporated by reference in its entirety.

This application was supported in part by ATP grant number NIST 70NANB3H3011. The government may have certain rights in the invention.

### FIELD OF THE INVENTION

The present invention relates generally to compositions and method for freezing and/or drying organs for storage prior to use. In particular, the present invention relates to the genetic modification of cells so that the cells themselves or tissues and organs formed from them can be dried.

### BACKGROUND

The emerging field of tissue engineering (TE) is poised to make enormous progress in treatment of organ disease and dysfunction in the coming decade. At present, there are 23 cell-based therapeutics approved for market in the United States and Europe, of which nine are skin substitutes or grafts, and 100 more products in development. De Bree, Genomics-based Drug Data Report and Regenerative Therapy (1)2:77-96 (2001). Overall the industry has had an annual growth rate of 16% from 1995-2001. The “structural” industry segment (e.g., skin, bone, cartilage) has shown 85% growth from 1998-2001.

Although a multitude of revolutionary and economically important applications for engineered tissues and organs exist in the human health arena, the full economic potential of the industry is far from realized. At present, only one of the publicly-held tissue engineering companies worldwide has shown a profit despite global invenstment in these technologies exceeding $3.5 billlion. Lysaght and Reyes, Tissue Engineering 7(5):485-93 (2001). Furthermore, in recent months set-backs in the industry resulted in cessation of operations or the withdrawal from market of at least two products (APLIGRAF from Organogenesis and DERMAGRAFT from Advanced Tissue Sciences).

A major impediment to the acceptance of engineered tissues by medical practitioners, healthcare providers, and second party payers is the lack of a means to effectively and efficiently preserve engineered tissues. The nature of living cells products makes them impractical for long-term storage. Current engineered tissues must often be stored and shipped under carefully controlled conditions to maintain viability and function. Typically, engineered tissue products take weeks or months to produce but must be used within hours or days after manufacture. As a result, engineered tissue companies must continually operate with their production facilities at top capacity and absorb the costs of inventory losses (i.e., unsold product which must be discarded). These inventory losses, on top of already costly manufacturing process, have forced prices to impracticable levels. As one specific example, APLIGRAF requires about four weeks to manufacture, is usable for less than six days and must be maintained between 20 and 37° C. until used. As another example, EPICEL is transported by a nurse from Cambridge, Mass. to the point-of-use in a portable incubator and is used immediately upon arrival. Such constraints represent significant challenges to developing convenient and cost-effective products.

Cryopreservation has been explored as a solution to the storage problem, but it is known to induce tissue damage through ice formation, chilling injury, and osmotic balance. Besides APLIGRAF, the only other approved skin equivalent, ORCEL, is currently in clinical trials as a frozen product but has the drawback that it must be maintained at temperatures below −100° C. prior to use. This means using liquid nitrogen storage, which is expensive, dangerous, and not universally available (e.g. rural clinics and field hospitals). Moreover, delivering a frozen product requires special training on the part of the end-user to successfully thaw the tissue prior to use.

Accordingly, what is needed in the art are improved engineered cells and tissues that are optimized for storage and methods of preparing engineered tissues and cells for long-term room temperature storage.

### SUMMARY OF THE INVENTION

The present invention relates generally to compositions and method for freezing and/or drying organs for storage prior to use. In particular, the present invention relates to the genetic modification of cells so that the cells themselves or tissues and organs formed from them can be dried. Accordingly, in some embodiments, the present invention provides a mammalian cell comprising a gene encoding a heterologous late embryogenesis abundant protein. The present invention is not limited to the use of any particular late embryogenesis abundant protein. Indeed, the use of a variety of late embryogenesis abundant proteins is contemplated, including Group 3 plant late embryogenesis abundant proteins. In some particularly preferred embodiments, the late embryogenesis abundant protein is HVA1. In other preferred embodiments, the cell is stably transfected with a late embryogenesis abundant protein gene. In further preferred embodiments, the late embryogenesis protein gene is operably linked to a promoter. The present invention is not limited to the use of any particular promoters. Indeed, a variety of promoters find use in the present invention including inducible and constitutive promoters. In still other preferred embodiments, the mammalian cell is a keratinocyte. In some embodiments, the keratinocyte is a primary keratinocyte. In other preferred embodiments, the cell is an immortalized keratinocyte. In particularly preferred embodiments, the keratinocyte is a NIKS cell. In other preferred embodiments, the NIKS cell is stratified. In still other embodiments, the cell is selected from the group consisting of stem cells, tissue culture cells, primary culture cells, and immortalized cells. In some embodiments, the cell is dried. The present invention is not limited to cells dried by any particular method. Indeed, the cells may be dried by a variety of methods including, but not limited to, freeze drying, air drying and vacuum drying.

In other preferred embodiments, the present invention provides a tissue or organ comprising a mammalian cell expressing a heterologous late embryogenesis abundant protein. The present invention is not limited to any particular organ. Indeed, the present invention contemplates the use of the cells to produce a variety of organs, including, but not limited to skin, heart, liver, pancreas, kidney and lung. In some preferred embodiments, the organ is a human skin equivalent. In still more preferred embodiments, the organ comprises NIKS cells. In other preferred embodiments, the organ comprises stratified NIKS cells. In some embodiments, the organ is dried. The present invention is not limited to organs dried by any particular method. Indeed, the organs may be dried by a variety of methods including, but not limited to, freeze drying, air drying and vacuum drying.

In still other embodiments, the present invention provides kits comprising a mammalian cell expressing heterologous late embryogenesis abundant protein and instructions for its use. In further embodiments, the present invention provides kits comprising an organ comprising mammalian cells expressing heterologous late embryogenesis abundant protein and instructions for its use.

In some embodiments, the present invention provides mammalian expression vectors comprising a gene encoding a plant late embryogenesis abundant protein operably linked to a promoter functional in mammalian cells. In some preferred embodiments, the late embryogenesis abundant protein is a Group 3 plant late embryogenesis abundant protein. In other preferred embodiments, the late embryogenesis protein is HVA1.

In some embodiments, the present invention provides a mammalian cell comprising a gene encoding a heterologous sugar (e.g., trehalose) transport protein. The present invention is not limited to the use of any particular trehalose transport protein. Indeed, the use of a variety of trehalose transport proteins is contemplated, including mutant, variant, and truncated trehalose transport protein. In some preferred embodiments, the trehalose transport protein is AGT1 (e.g., the AGT1 of SEQ ID NO:3). In other preferred embodiments, the cell is stably transfected with the trehalose transport protein. In further preferred embodiments, the trehalose transport protein is operably linked to a promoter. The present invention is not limited to the use of any particular promoters. Indeed, a variety of promoters find use in the present invention including inducible and constitutive promoters. In still other preferred embodiments, the mammalian cell is a keratinocyte. In some embodiments, the keratinocyte is a primary keratinocyte. In other preferred embodiments, the cell is an immortalized keratinocyte. In particularly preferred embodiments, the keratinocyte is a NIKS cell. In other preferred embodiments, the NIKS cell is stratified. In still other embodiments, the cell is selected from the group consisting of stem cells, tissue culture cells, primary culture cells, and immortalized cells. In some embodiments, the cell is dried. The present invention is not limited to cells dried by any particular method. Indeed, the cells may be dried by a variety of methods including, but not limited to, freeze drying, air drying and vacuum drying.

In other preferred embodiments, the present invention provides a tissue or organ comprising a mammalian cell expressing a heterologous trehalose transport protein. The present invention is not limited to any particular organ. Indeed, the present invention contemplates the use of the cells to produce a variety of organs, including, but not limited to skin, heart, liver, pancreas, kidney and lung. In some preferred embodiments, the organ is a human skin equivalent. In still more preferred embodiments, the organ comprises NIKS cells. In other preferred embodiments, the organ comprises stratified NIKS cells. In some embodiments, the organ is dried. The present invention is not limited to organs dried by any particular method. Indeed, the organs may be dried by a variety of methods including, but not limited to, freeze drying, air drying and vacuum drying.

In still other embodiments, the present invention provides kits comprising a mammalian cell expressing heterologous trehalose transport protein and instructions for its use. In further embodiments, the present invention provides kits comprising an organ comprising mammalian cells expressing heterologous trehalose transport protein and instructions for its use.

In some embodiments, the present invention provides mammalian expression vectors comprising a gene encoding a trehalose transport protein operably linked to a promoter functional in mammalian cells.

In some embodiments, the present invention provides a cell comprising genes encoding a heterologous trehalose synthesis pathway. In some embodiments, the genes encoding a trehalose synthesis pathway comprise otsA and otsB. In some preferred embodiments, the otsA has the nucleic acid sequence of SEQ ID NO: 7 and the otsB has the nucleic acid sequence of SEQ ID NO: 6. In other preferred embodiments, the keratinocyte is stably transfected with the heterologous trehalose synthesis pathway genes. In further preferred embodiments, the genes encoding a heterologous trehalose synthesis pathway are operably linked to a promoter. The present invention is not limited to the use of any particular promoter. Indeed, a variety of promoters find use in the present invention including inducible and constitutive promoters. In some embodiments, the otsA and otsB genes are in the same expression vector. In other embodiments, they are on two different expression vectors. In some preferred embodiments, the otsA and otsB genes are on two different expression vectors and the expression vectors are present at a ratio of 2 otsA containing vectors to one otsB containing vector. In some embodiments, otsA and otsB gene functions are contained on one gene and/or one transcript. In some embodiments, the cell is a keratinocyte. In some embodiments, the keratinocyte is a primary keratinocyte. In other preferred embodiments, the keratinocyte is an immortalized keratinocyte. In particularly preferred embodiments, the keratinocyte is a NIKS cell. In other preferred embodiments, the NIKS cell is stratified. In still other embodiments, the cell is selected from the group consisting of stem cells, tissue culture cells, primary culture cells, and immortalized cells. In some embodiments, the cell is dried. The present invention is not limited to cells dried by any particular method. Indeed, the cells may be dried by a variety of methods including, but not limited to, freeze drying, air drying and vacuum drying.

In other preferred embodiments, the present invention provides a tissue or organ comprising a mammalian cell expressing genes encoding a heterologous trehalose synthesis pathway. The present invention is not limited to any particular organ. Indeed, the present invention contemplates the use of the cells to produce a variety of organs, including, but not limited to skin, heart, liver, pancreas, kidney and lung. In some preferred embodiments, the organ is a human skin equivalent. In still more preferred embodiments, the organ comprises NIKS cells. In other preferred embodiments, the organ comprises stratified NIKS cells. In some embodiments, the organ is dried. The present invention is not limited to organs dried by any particular method. Indeed, the organs may be dried by a variety of methods including, but not limited to, freeze drying, air drying and vacuum drying.

In still other embodiments, the present invention provides kits comprising a mammalian cell expressing heterologous genes encoding a heterologous trehalose synthesis pathway and instructions for its use. In further embodiments, the present invention provides kits comprising an organ comprising mammalian cells expressing heterologous trehalose transport protein and instructions for its use.

In some embodiments, the present invention provides mammalian expression vectors comprising genes encoding a heterologous trehalose synthesis pathway operably linked to a promoter functional in mammalian cells.

In some embodiments, the present invention provides methods of preserving mammalian cells comprising a) providing cells comprising a gene encoding a plant late embryogenesis abundant protein; b) culturing said cells under conditions such that said gene encoding a plant late embryogenesis abundant protein is expressed; and c) freezing said mammalian cells. In some embodiments, the methods further comprise step d) drying said cells. The present invention is not limited to the use of any particular late embryogenesis abundant protein. Indeed, the use of a variety of late embryogenesis abundant proteins is contemplated, including Group 3 plant late embryogenesis abundant proteins. In some particularly preferred embodiments, the late embryogenesis abundant protein is HVA1. In other preferred embodiments, the cell is stably transfected with the late embryogenesis abundant protein. In further preferred embodiments, the late embryogenesis protein gene is operably linked to a promoter. The present invention is not limited to the use of any particular promoters. Indeed, a variety of promoters find use in the present invention including inducible and constitutive promoters. In still other preferred embodiments, the mammalian cell is a keratinocyte. In some embodiments, the keratinocyte is a primary keratinocyte. In other preferred embodiments, the cell is an immortalized keratinocyte. In particularly preferred embodiments, the keratinocyte is a NIKS cell. In other preferred embodiments, the NIKS cell is stratified. In still other embodiments, the cell is selected from the group consisting of stem cells, tissue culture cells, primary culture cells, and immortalized cells. The present invention is not limited to drying by any particular method. Indeed, the cells may be dried by a variety of methods including, but not limited to, freeze drying, air drying and vacuum drying.

In some embodiments, the cells are incorporated into an organ. In some preferred embodiments, the organ is skin. In other preferred embodiments, the organ is a human skin equivalent. In some embodiments, the organ comprises NIKS cells. In other preferred embodiments, the organ comprises stratified NIKS cells.

In some embodiments, the present invention provides methods of preserving mammalian cells comprising a) providing cells comprising a gene encoding a trehalose transport protein (e.g., including, but not limited to, AGT1); b) culturing said cells under conditions such that said gene encoding a trehalose transport protein is expressed; c) exposing cells to trehalose under conditions such that trehalose is taken into the cells by the transport protein; and d) freezing said mammalian cells. In some preferred embodiments, the cell is stably transfected with the trehalose transport protein. In further preferred embodiments, the trehalose transport protein gene is operably linked to a promoter. The present invention is not limited to the use of any particular promoters. Indeed, a variety of promoters find use in the present invention including inducible and constitutive promoters. In some embodiments, the exposing step is performed at a pH of about 5.5 or lower. In still other preferred embodiments, the mammalian cell is a keratinocyte. In some embodiments, the keratinocyte is a primary keratinocyte. In other preferred embodiments, the cell is an immortalized keratinocyte. In particularly preferred embodiments, the keratinocyte is a NIKS cell. In other preferred embodiments, the NIKS cell is stratified. In still other embodiments, the cell is selected from the group consisting of stem cells, tissue culture cells, primary culture cells, and immortalized cells. The present invention is not limited to drying by any particular method. Indeed, the cells may be dried by a variety of methods including, but not limited to, freeze drying, air drying and vacuum drying.

In some embodiments, the cells are incorporated into an organ. In some preferred embodiments, the organ is skin. In other preferred embodiments, the organ is a human skin equivalent. In some embodiments, the organ comprises NIKS cells. In other preferred embodiments, the organ comprises stratified NIKS cells.

In some embodiments, the present invention provides methods of preserving mammalian cells comprising a) providing cells comprising genes encoding a trehalose synthesis pathway; b) culturing said cells under conditions such that said genes encoding a trehalose synthesis pathway are expressed and the cells take up trehalose; and c) freezing said mammalian cells. In some embodiments, the genes encoding a trehalose synthesis pathway comprise otsA and otsB. In some preferred embodiments, the otsA has the nucleic acid sequence of SEQ ID NO: 7 and the otsB has the nucleic acid sequence of SEQ ID NO: 6. In some preferred embodiments, the cell is stably transfected with the trehalose transport protein. In further preferred embodiments, the genes encoding a trehalose synthesis pathway are operably linked to a promoter. The present invention is not limited to the use of any particular promoters. Indeed, a variety of promoters find use in the present invention including inducible and constitutive promoters. In some embodiments, the otsA and otsB genes are in the same expression vector. In other embodiments, they are on two different expression vectors. In some preferred embodiments, the otsA and otsB genes are on two different expression vectors and the expression vectors are present at a ratio of 2 otsA containing vectors to one otsB containing vector. In some embodiments, otsA and otsB gene functions are contained on one gene and/or one transcript. In still other preferred embodiments, the mammalian cell is a keratinocyte. In some embodiments, the keratinocyte is a primary keratinocyte. In other preferred embodiments, the cell is an immortalized keratinocyte. In particularly preferred embodiments, the keratinocyte is a NIKS cell. In other preferred embodiments, the NIKS cell is stratified. In still other embodiments, the cell is selected from the group consisting of stem cells, tissue culture cells, primary culture cells, and immortalized cells. The present invention is not limited to drying by any particular method. Indeed, the cells may be dried by a variety of methods including, but not limited to, freeze drying, air drying and vacuum drying.

In some embodiments, the cells are incorporated into an organ. In some preferred embodiments, the organ is skin. In other preferred embodiments, the organ is a human skin equivalent. In some embodiments, the organ comprises NIKS cells. In other preferred embodiments, the organ comprises stratified NIKS cells.

In still further embodiments, the cells comprise combinations of heterologous trehalose transport protein, late embryogenesis abundant protein and trehalose synthesis pathway genes.

In still other embodiments, the present invention provides methods of freezing mammalian cells comprising: a) providing immortalized keratinocyte cells, wherein said cells both contain trehalose and are treated extracellularly with trehalose; b) treating said cells with an oxyanion; c) and freezing said cells. In some embodiments, the methods further comprise d) drying said cells. In some embodiments, the oxyanion is phosphate. In some preferred embodiments, the keratinocytes are NIKS cells. In further preferred embodiments, the NIKS cells are stratified. In other preferred embodiments, the NIKS cell is stratified. The present invention is not limited to drying by any particular method. Indeed, the cells may be dried by a variety of methods including, but not limited to, freeze drying, air drying and vacuum drying.

In some embodiments, the present invention provides methods of treating a patient comprising: a) providing a patient suffering from a condition and an organ preserved by drying; b) treating said patient with said organ preserved by drying under conditions such that said condition is relieved. In some embodiments, the organ is freeze dried. In other embodiments, the organ is air or vacuum dried. In further embodiments, the organ comprises keratinocytes. In some preferred embodiments, the keratinocytes are NIKS cells. In other preferred embodiments, the NIKS cells are stratified. In some embodiments, the organ is a human skin equivalent. In still other embodiments, the patient is suffering from a condition selected from the group consisting of a burn, wound, donor site wound, and ulcer. In some embodiments, the organ comprises cells expressing an exogenous trehalose transporter protein. In other embodiments, the organ comprises cells expressing a plant late embryogenesis abundant. In still other embodiments, the plant late embryogenesis protein is HVA1. In further embodiments, the organ comprises cells expressing a trehalose synthesis pathway.

In other embodiments, the present invention provides a method of preserving mammalian cells comprising providing cells comprising a gene encoding a trehalose synthesis pathway; culturing the cells under conditions such that the cells comprise intracellular trehalose at a concentration of at least 5 mM; and freezing the mammalian cells.

### DESCRIPTION OF FIGURES

FIG. 1 is a solution phase diagram.

FIG. 2 provides the sequence for HVA1.

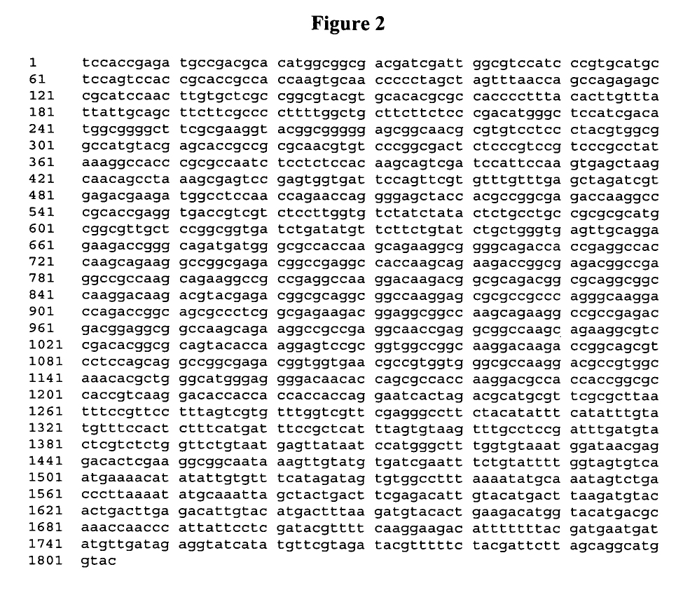

FIG. 3 provides the sequence for trehalose transport protein AGT1.

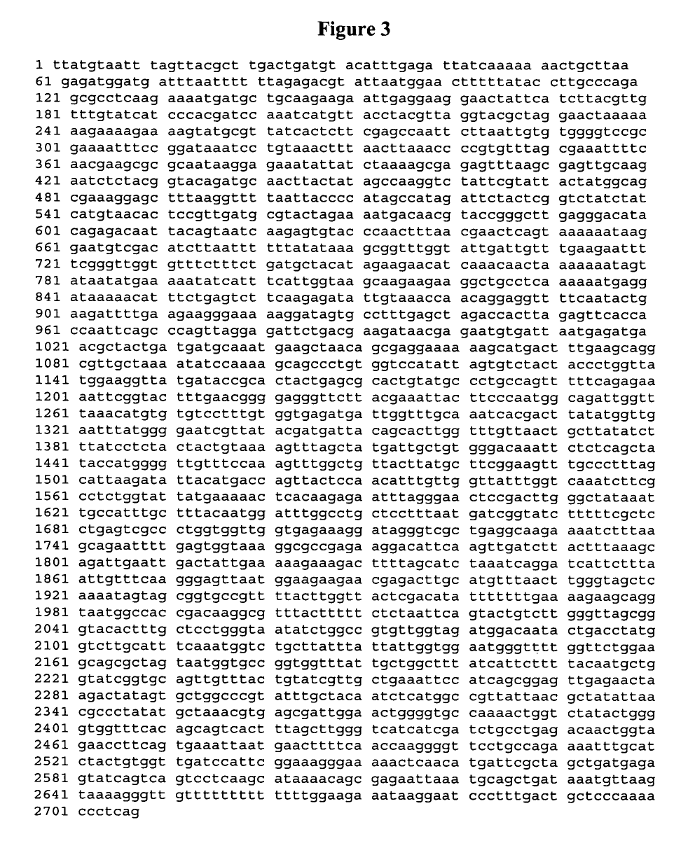

FIG. 4 provides the sequence for otsA.

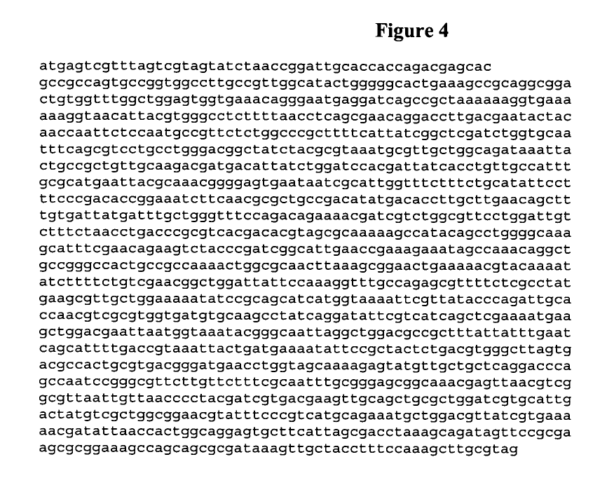

FIG. 5 provides the sequence for otsB.

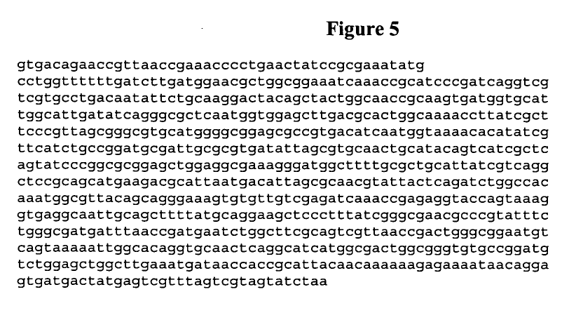

FIG. 6 presents a RT-PCR result for otsB gene expression in NIKS cells.

FIG. 7 shows splicing patterns for otsB and otsA.

FIG. 8 presents a RT-PCR result for mutated otsB gene expression in NIKS cells.

FIG. 9 presents a RT-PCR result for otsA gene expression in NIKS cells.

FIG. 10 presents a RT-PCR result for mutated otsA gene expression in NIKS cells.

FIG. 11 shows an agarose gel demonstrating the RT-PCR result for AGT1 mRNA expression.

FIG. 12 shows the results of differing levels of the otsA construct relative to otsB on trehalose synthesis.

FIG. 13 shows the results of HPLC analysis of trehalase digestion.

FIG. 14 shows the results of trehalose uptake at different pHs.

FIG. 15 shows the effect of NIKS cell lysate on pNPαG in solution.

FIG. 16 shows vectors for the constitutive expression of otsA and otsB.

FIG. 17 shows a map of pTRE-tight-AGT1-hyg vector and the hygromycin cassette.

FIG. 18 shows the sequences of mutated otsA (SEQ ID NO:7) and otsB (SEQ ID NO:6).

FIG. 19 shows the levels of trehalose in mixtures of cells comprising an otsA expressing vector and cells comprising an otsB expressing vector.

### DEFINTIONS

As used herein, the terms “human skin equivalent” and “human skin substitute” are used interchangeably to refer to an in vitro derived culture of keratinocytes that has stratified into squamous epithelia. Typically, the skin equivalents are produced by organotypic culture.

As used herein, the term “late embryogenesis abundant protein” when used in reference to a protein or nucleic acid encoding a protein refers to a class of hydrophilic proteins that are produced in plants during late embryogenesis (e.g., SEQ ID NO: 1). Thus, the term late embryogenesis abundant protein encompasses both proteins that are identical to wild-type late embryogenesis abundant proteins and those that are derived from wild type late embryogenesis abundant proteins (e.g., variants of late embryogenesis abundant proteins, chimeric genes constructed with portions of late embryogenesis abundant protein coding regions, or humanized late embryogenesis abundant proteins).

As used herein, the term “Group 3 late embryogenesis abundant protein” when used in reference to a protein or nucleic acid encoding a protein refer to a class of hydrophilic proteins that are produced in plants during late embryogenesis. This term includes proteins characterized by an 11 amino acid motif: apolar-apolar-neg./amide-X-apolar-positive-negative-positive-apolar-X-basic, an example of which is TAQAAKEKAGE (SEQ ID NO:2). Thus, the term Group 3 late embryogenesis abundant protein encompasses both proteins that are identical to wild-type Group 3 late embryogenesis abundant proteins and those that are derived from wild type Group 3 late embryogenesis abundant proteins (e.g., variants of Group 3 late embryogenesis abundant proteins, chimeric genes constructed with portions of Group 3 late embryogenesis abundant protein coding regions, or humanized Group 3 late embryogenesis abundant proteins).

As used herein, the term “HVA1” when used in reference to a protein or nucleic acid refers to a protein or nucleic acid encoding a protein that shares greater than about 50% identity with SEQ ID NO: 1 and also has at least one activity of wild type HVA1. Thus, the term HVA1 protein encompasses both proteins that are identical to wild-type HVA1 protein and those that are derived from wild type HVA1 protein (e.g., variants of HVA1 protein, chimeric genes constructed with portions of HVA1 protein coding regions, or humanized HVA1 proteins).

As used herein, the term “activity of HVA1” refers to any activity of wild type HVA1 protein. The term is intended to encompass all activities of HVA1 protein, alone or in combination.

In particular, the term “HVA1 gene” refers to the full-length HVA1 nucleotide sequence (e.g., contained in SEQ ID NO:1). However, it is also intended that the term encompass fragments of the HVA1 sequence, as well as other domains within the full-length HVA1 nucleotide sequence, as well as variants of HVA1. Furthermore, the terms “HVA1 gene nucleotide sequence” or “HVA1 gene polynucleotide sequence” encompasses DNA, cDNA, and RNA (e.g., mRNA) sequences.

As used herein, the term “trehalose transport protein” when used in reference to a protein or nucleic acid refers to a protein or nucleic acid encoding a protein that shares greater than about 50% identity with SEQ ID NO:3 and also has at least one activity of wild type trehalose transport protein (e.g., binding to a trehalose). Such binding can be assayed by standard methodologies such as ELISA. Thus, the term trehalose transport protein encompasses both proteins that are identical to wild-type trehalose transport protein and those that are derived from wild type trehalose transport protein (e.g., variants of trehalose transport protein or chimeric genes constructed with portions of trehalose transport protein coding regions).

As used herein, the term “activity of trehalose transport protein” refers to any activity of wild type trehalose transport protein. The term is intended to encompass all activities of trehalose transport protein, alone or in combination.

In particular, the term “trehalose transport protein gene” refers to the full-length trehalose transport protein AGT1 nucleotide sequence (e.g., contained in SEQ ID NO:3). However, it is also intended that the term encompass fragments of the trehalose transport protein sequence, as well as other domains within the full-length trehalose transport protein nucleotide sequence, as well as variants of trehalose transport protein. Furthermore, the terms “trehalose transport protein gene nucleotide sequence” or “trehalose transport protein gene polynucleotide sequence” encompasses DNA, cDNA, and RNA (e.g., mRNA) sequences.

As used herein, the term “trehalose synthesis pathway” when used in reference to a proteins or nucleic acids encoding proteins refers to proteins that are necessary for synthesizing trehalose. The term trehalose synthesis pathway protein encompasses both proteins that are identical to wild-type trehalose synthesis pathway proteins and those that are derived from wild type trehalose synthesis pathway proteins (e.g., variants of trehalose synthesis pathway proteins, chimeric genes constructed with portions of trehalose synthesis pathway protein coding regions, or humanized trehalose synthesis pathway proteins).

As used herein, the term “otsA” when used in reference to a protein or nucleic acid refers to a protein or nucleic acid encoding a protein that shares greater than about 50% identity with SEQ ID NO:4 and also has at least one activity of wild type otsA. Such activity can be assayed by standard methodologies such as colorimetric assays. Thus, the term otsA protein encompasses both proteins that are identical to wild-type otsA protein and those that are derived from wild type otsA protein (e.g., variants of trehalose synthesis protein (e.g., SEQ ID NO:7), chimeric genes constructed with portions of trehalose synthesis protein coding regions, or humanized otsA).

As used herein, the term “activity of otsA” refers to any activity of wild type otsA protein. The term is intended to encompass all activities of otsA protein, alone or in combination.

In particular, the term “otsA gene” refers to the full-length otsA nucleotide sequence (e.g., contained in SEQ ID NO:4). However, it is also intended that the term encompass fragments of the otsA sequence, as well as other domains within the full-length otsA nucleotide sequence, as well as variants of otsA. Furthermore, the terms “otsA gene nucleotide sequence” or “otsA gene polynucleotide sequence” encompasses DNA, cDNA, and RNA (e.g., mRNA) sequences.

As used herein, the term “otsB” when used in reference to a protein or nucleic acid refers to a protein or nucleic acid encoding a protein that shares greater than about 50% identity with SEQ ID NO:5 and also has at least one activity of wild type otsB. Such activity can be assayed by standard methodologies such as colorimetric assays. Thus, the term otsA protein encompasses both proteins that are identical to wild-type otsB protein and those that are derived from wild type otsB protein (e.g., variants of trehalose synthesis protein (e.g., SEQ ID NO:6), chimeric genes constructed with portions of trehalose synthesis protein coding regions, or humanized otsB).

As used herein, the term “activity of otsB” refers to any activity of wild type otsB protein. The term is intended to encompass all activities of otsB protein, alone or in combination.

In particular, the term “otsB gene” refers to the full-length otsB nucleotide sequence (e.g., contained in SEQ ID NO:5). However, it is also intended that the term encompass fragments of the otsB sequence, as well as other domains within the full-length otsB nucleotide sequence, as well as variants of otsB. Furthermore, the terms “otsB gene nucleotide sequence” or “otsB gene polynucleotide sequence” encompasses DNA, cDNA, and RNA (e.g., mRNA) sequences.

As used herein, the term “vitrification” refers to the process of freezing a sample at a rate fast enough to substantially prevent ice crystal formation.

As used herein, the term “freeze drying” refers to the sublimation of water from a sample.

As used herein, the term “air drying” refers to drying caused by exposure to air or some other gas.

As used herein, the term “vacuum drying” refers to the removal of moisture by exposure to a vacuum.

As used herein, the term “NIKS cells” refers to cells having the characteristics of the cells deposited as cell line ATCC CRL-1219.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements. As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described below.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention (e.g., trehaolase transport protein).

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “gene” refers to a nucleic acid (e.g., DNA) sequence that comprises coding sequences necessary for the production of a polypeptide or precursor (e.g., KGF-2). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “recombinant DNA molecule” as used herein refers to a DNA molecule that is comprised of segments of DNA joined together by means of molecular biological techniques.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding trehalose binding protein includes, by way of example, such nucleic acid in cells ordinarily expressing trehalose binding protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets that specify stop codons (i.e., TAA, TAG, TGA).

As used herein, the term “purified” or “to purify” refers to the removal of contaminants from a sample.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

“Operably linked” refers to a juxtaposition wherein the components so described are in a relationship permitting them to function in their intended manner. A regulatory sequence is “operably linked” to a coding sequence when it is joined in such a way that expression of the coding sequence is achieved under conditions compatible with the regulatory sequence.

“PCR” refers to the techniques of the polymerase chain reaction as described in Saiki, et al., Nature 324:163 (1986); and Scharf et al., Science 233:1076-1078 (1986); U.S. Pat. No. 4,683,195; and U.S. Pat. No. 4,683,202.

By “pharmaceutically acceptable carrier,” is meant any carrier that is used by persons in the art for administration into a human that does not itself induce any undesirable side effects such as the production of antibodies, fever, etc. Suitable carriers are typically large, slowly metabolized macromolecules that can be a protein, a polysaccharide, a polylactic acid, a polyglycolic acid, a polymeric amino acid, amino acid copolymers or an inactive virus particle. Such carriers are well known to those of ordinary skill in the art. Preferably the carrier is thyroglobulin.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the GKLF mRNA-specific signal observed on Northern blots). The amount of mRNA present in the band corresponding in size to the correctly spliced trehalose binding protein transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the term “response”, when used in reference to an assay, refers to the generation of a detectable signal (e.g., accumulation of reporter protein, increase in ion concentration, accumulation of a detectable chemical product).

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

### GENERAL DESCRIPTION

Three issues stand out in the field of preservation of organs. First, both intra- and extra-cellular vitrification using trehalose have demonstrated benefits for dry storage of isolated cells. Second, this benefit is not manifest in all cases, indicating other important aspects to this problem exist. Third, there has never been reported drying success for tissues or organs. As described below, the present invention provides solutions to these problems.

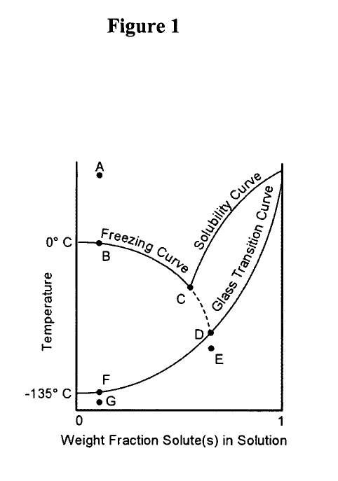

Slow freezing is currently the universal industrial choice for the preservation and storage of living mammalian cells and tissues. The solution phase diagram for a simple system is given in FIG. 1. A tissue in culture medium is first cooled (A). When the freezing point of water is reached (B), ice begins to crystallize, increasing the concentration of the solutes remaining in solution, so-called “freeze-concentration.” This process continues until the solutes crystallize at the eutectic point (C). Multi-component systems, however, rarely behave in this ideal fashion. Usually, and especially in the case of many cryoprotectant solutions, the solutes do not crystallize, and the solution supersaturates until it forms a glass where no further crystallization takes place (D). At this point the glassy material can be cooled with no further change in composition (point E or lower).

Three main mechanisms exist by which damage occurs in tissues and organs during slow freezing; extracellular ice crystal formation, osmotic imbalance, and chill injury. Pegg, Cryo-Letters 22(2):105-114 (2001). Extracellular ice formation is capable of disrupting fine organ structure, a problem unique to tissue and organ preservation. Furthermore, freeze-concentration effects lead to many damaging processes most prominent among them being cell volume changes. As the extracellular spaces become hypertonic, cells lose water to avoid osmotic imbalance. This shrinkage gives rise to cytoskeletal damage, cell leakage, and membrane fusion. Thawing tissues causes the reverse effect, cell swelling and rupture. Chill injury is a result of metabolic changes due to temperature reduction itself such as expression of heat shock proteins or apoptosis. Liu et al., Tissue Engineering 6(5):539-54 (2000); Baust et al., In Vitro Cellular & Developmental Biology-Animal 36(4):262-270 (2000); Fowke et al., J. Immunol. Meth. 244 (1-2):139-144 (2000). It is worth noting that all of these effects are reduced by rapid freezing.

The current state of the art for mammalian tissue and organ cryopreservation focuses on minimization of osmotic effects. To improve survival of a tissue through freezing, various compounds are added as ‘osmoprotectants,’ most commonly glycerol and dimethylsulfoxide (DMSO). These compounds readily cross the cell membrane increasing a cell's internal osmotic pressure thereby reducing its volume fluctuations and improving its chances for survival. Despite their effectiveness, glycerol and DMSO cannot be used to store cells at temperatures greater than −100° C., the approximate T_{g} of these systems, due to the greatly increased rates of cell and tissue damage. Furthermore, these compounds are toxic to cells, a fact that limits their utility at production scale.

One alternative to traditional osmoprotection is exemplified by yeast, which synthesize an intracellular protectant as needed. The favored protectant for yeast and many other desiccation-resistant organisms is the sugar trehalose. Leopold, ed. Membranes, metabolism, and Dry Organisms, Cornell Univ. Press, Ithaca (1986). Trehalose is a non-reducing disaccharide of glucose. Its ability to form glasses and mimic the hydrogen bonding character of water in addition to its osmoprotectant qualities and chemical stability makes it well-suited for our proposed work. It has been shown to stabilize dried lipid vesicles, sperm, and murine embryos. Crowe et al., Biochim. Biophys. Acta 769:141-150 (1984); Storey et al., Cryobiology 37(1):46-58 (1998); Ishida et al., Human Repro. 12(6):1259-62 (1997). In most cases trehalose's protectant ability is superior to other saccharides, but it can be improved upon via the novel approaches to solution engineering described below. Crowe et al., Cryobiology 43(2):89-105 (2001).

As mentioned above, damage to organs during preservation includes ice formation, osmotic effects, and chill injury. Rapid freezing or vitrification can overcome these barriers. As depicted by path A→B→F→G in FIG. 1, vitrification is the process of freezing a sample at a rate fast enough to prevent ice crystal formation. By forming a glass from the starting solution, no ice crystals form, solute concentrations do not increase, cell shrinkage is avoided, and osmoprotection is unnecessary. Thus, vitrification eliminates nearly all of the complications associated with slow freezing of tissues and organs; moreover, the speed of freezing during vitrification is great enough that chill injury effects such as induction of apoptosis are minimized. Borderie et al., Invest. Ophthal & Visual Sci. 39(8):1511-19 (1998). If appropriate vitrification solutions are used (see below), such a process will be highly scalable and will accommodate many product types.

A significant challenge in applying vitrification to the industrial preservation of tissues, however, is the prevention of intracellular ice crystal formation (IIF). Toner et al., AIChE Jornal 38(10):1512-22 (1992). This lethal effect arises since cells do not dehydrate during vitrification and retain relatively high levels of cytosolic water. Muldrew et al., Biophys. J. 66:532-41 (1994). One approach to this challenge is introducing trehalose to the interior of cells, where it increases the T_{g} of the cytosol and reduces or eliminates the likelihood of IIF. It has recently been shown in the cases of isolated human pancreatic islets, suspended fibroblasts and keratinocytes that intracellular trehalose improves recovery of viable cells after freezing. Beattie et al., Diabetes 46:519-23 (1997); Eroglu et al., Nature Biotech. 18(2):163-67 (2000).

While intracellular trehalose minimizes IIF, it does not cross the cell membrane, and innovative strategies are needed to achieve this effect. The present invention provides novel methods of introducing trehalose into mammalian cells, including engineering the cells to express a trehalose transport protein and/or to express components of the trehalose synthesis pathway. These methods and compositions are described in more detail below.

To date, trehalose has been introduced into isolated mammalian cells using biophysical phenomena, insertion of genes for trehalose synthesis enzymes, and exogenous pore forming proteins. Eroglu et al., supra; Guo et al., Nature Biotech. 18(2):168-71 (2000); Lao et al., Cryobiology 42(3):207-17 (2001). The first reported biophysical approach made use of the liquid crystal to gel membrane phase transition in isolated pancreatic islet cells. Beattie et al., supra. At this transition point, cell membranes become permeable to small molecules such as trehalose. These workers found that cryopreserving islet cells in this manner doubled the number of viable cells recovered. More recently intracellular trehalose delivery has been achieved using heat shock treatments and inducing endocytosis. Puhlev et al., Cryobiology 42(3):207-17 (2001); Wolkers et al., Cryobiology 42(2):79-87 (2001). Complications, however, will arise in the application of these techniques to tissues and organs. For instance, since organs are comprised of multiple cell types each with its own transition temperature, optimal recovery of all cells is impossible.

Addition of intracellular trehalose has not in all cases shown benefit in drying cells. Thus, the present invention also contemplates the introduction of plant late embryogenesis abundant (LEA) proteins into mammalian cells. LEA proteins are class of hydrophilic proteins found in plants with homologous proteins in a wide variety of organisms including humans. Shen et al., Plant Mol. Biol. 45(3):327-40 (2001). LEAs are grouped into classes based on the amino acid sequence of repeat motifs that appear to be important in their function. Dure et al., Plant Molec. Biol. 12(5):475-86 (1989). The present invention particularly contemplates the use of Group 3 Lea proteins, which have an 11 amino acid repeat that is similar to antifreeze proteins found in freeze-tolerant fish. Holmberg et al., Trends Plant Sci. 3(2)″61-66 (1998). The appearance of these LEAs is correlated with the accumulation of trehalose in some nematodes. Solomon et al., Parasitology 121:409-16 (2000). Some Group 3 LEAs have been shown to be highly effective cryostabilizers. Honjoh et al., Biosci. Biotech. Biochem. 64(8):1656-63 (2000). The function of these proteins is believed to hinge on their strong interaction with sugars resulting in increased cytoplasmic T_{g}s. Wolkers et al., Biochim. Biophys. Acta—Prot. Structure and Mol. Enzym. 1544(1-2):196-206 (2001).

The present invention particularly contemplates the engineering of mammalian cells with the HVA1 gene from barley (Hordeum vulgare), a Group 3 LEA. The HVA1 protein has been found at roughly 1% total protein during seed maturation. Straub et al., Plant Mol. Biol. 26(2):617-30 (1994). By comparison, the protein actin in human cells is expressed at a level of roughly 5% total protein. The HVA1 gene has been cloned into and expressed in other nonmammalian species resulting in improvements in dehydration tolerance. Zhang et al., J. Biochem. 127(4):611-16 (2000); Xu et al., Plant Phys. 110(1):249-57 (1996).

The present invention is not limited to any particular mechanism. Indeed, an understanding of the mechanism of the present invention is not necessary. Nevertheless, it is believed that the HVA1 protein enhances intracellular vitrification in concert with trehalose in several ways. First, LEAs have been shown to have strong binding interaction with sugars, which may enable them to provide a scaffold on which trehalose's vitrification behavior will be amplified. Second, the repeat units of the protein are hydrophilic allowing them to retain moisture during dehydration and to “replace water” in extreme desiccation. Third, the repeat units are believed to interact between proteins allowing for the formation of a protein network that mechanically stabilizes the cell. Finally, the HVA1 protein is well suited to binding phosphate ions. Dure et al., Plant J. 3(3):363-69 (1993). In the following disclosure it is shown that phosphate also interacts with trehalose. Thus, it is reasonable to propose that a long range ordering may take place between trehalose, phosphate, and LEA proteins during desiccation.

For successful organ preservation the extracellular spaces must remain free of ice. Fahy et al., Cryobiology 21: 407-26 (1984). This is problematic since these compartments have higher water contents and lower T_{g}s (points G versus E in FIG. 1). To reduce the tendency to form extracellular ice, glass-enhancing agents (e.g., sugars or polymers) can be added to achieve vitrification at more moderate cooling rates. Miller et al., Pharm. Res. 14(5):578-90 (1997); Crowe et al., Crybiology 35(1):20-30 (1997). The field of vitrifying organs has made advances in recent years, but the reported studies have used high concentrations of protectants with low T_{g}s resulting in cryogenic storage requirements. It is the goal of this work to develop solutions capable of ambient storage. Song et al., J. Invest. Surg. 13(5):279-88 (2000); Wowk et al., Cryobiology 40(3):228-36 (1997); Brockbank et al., Trans. Proc. 32(1):3-4 (2000). It is contemplated that for the purposes herein, trehalose is an excellent vehicle for extracellular vitrification and drying.

Despite its excellent glass-forming abilities, recent work has shown that the T_{g} of trehalose solutions can be dramatically increased. For many years it has been known that borate ions will form crosslinked complexes with polyhydroxy compounds. It was not discovered until recently, however, that the same chemistry applies to mixtures of borate ions with trehalose. Miller et al., J. Phys. Chem. B. 103(46):10243 (1999). Trehalose-borate was found to be a far superior freezing and freeze-drying protectant for protein and bacterial systems. Miller et al., Phar, Res. 15(8):1215-21 (1998). The use of borate for tissue preservation, however, is suboptimal due to its high pH and toxicity. Thus, phosphate was identified as an alternative crosslinking agent. Hasjim et al., Pharm. Res. 2000.

In a vitrification process thawing becomes a significant source of damage due to recrystallization during warming. The two most straightforward means of circumventing this phenomenon is to dry the sample before recrystallization takes place (freeze-drying) or to dry the sample directly without freezing (air drying).

In traditional freeze-drying, acceptable rates of water removal are achieved as a consequence of the porosity of the material remaining after sublimation of ice crystals. In a vitrified system, no ice crystals exist; the sample is a solid matrix from which water diffusion will be relatively slow. In order to counteract the effects of reduced porosity, a cost-effective drying cycle can be obtained if the glass transition temperature (and therefore the maximum drying temperature) can be raised to increase the rate of water removal. The present invention solves this problem by providing vitrification solutions with extraordinarily high T_{g}'s based on trehalose-oxyanion crosslinking enabling the production of fully dried, vitrified samples at reasonable cost.

The present invention also provides methods of producing a room temperature-stable tissue product by ambient air drying. There have been several recent reports of air drying mammalian cells. Wolkers et al., Cryobiology 42(2):79-87 (2001); Guo et al., Nature Biotech. 18(2):168-171 (2000); Puhlev et al., Cryobiology 42(3):207-17 (2001); Gordon et al., Cryobiology 43(2):114-123 (2000); Tablin et al., Cryobiology 43(2):114-23 (2001). It is contemplated that the intracellular synthesis of both trehalose and the HVA1 LEA protein will allow us to develop systems for dry storage that are more robust and reproducible than those of other workers.

### DETAILED DESCRIPTION

The present invention relates generally to compositions and method for freezing and/or drying organs for storage prior to use. In particular, the present invention relates to the genetic modification of cells so that the cells themselves or tissues and organs formed from them can be dried. For convenience, the description of the invention is presented in the following sections: A) Genetic Modification and Optimization of Cells for Freezing and Drying; B) Modified Cell Lines; C) Production of Organs and Tissues; D) Preservation of Organs and Tissues; and E) Therapuetic Uses.

A) Genetic Modification and Optimization of Cells for Freezing and Drying

In preferred embodiments, mammalian cells (e.g., NIKS cells) expressing genes for optimization of freezing and drying can be produced by conventional gene expression technology using methods well-known in the art, as discussed in more detail below. The practice of the present invention will employ, unless otherwise indicated, conventional techniques of molecular biology, microbiology, recombinant DNA, and immunology, which are within the skill of the art. Such techniques are explained fully in the literature, including Sambrook, et al., Molecular Cloning: A Laboratory Manual 2nd ed. (Cold Spring Harbor Laboratory Press, 1989); DNA Cloning, Vol. I and II, D. N Glover ed. (IRL Press, 1985); Oligonucleotide Synthesis, M. J. Gait ed. (IRL Press, 1984); Nucleic Acid Hybridization, B. D. Hames & S. J. Higgins eds. (IRL Press, 1984); Transcription and Translation, B. D. Hames & S. J. Higgins eds., (IRL Press, 1984); Animal Cell Culture, R. I. Freshney ed. (IRL Press, 1986); Immobilized Cells and Enzymes, K. Mosbach (IRL Press, 1986); B. Perbal, A Practical Guide to Molecular Cloning, Wiley (1984); the series, Methods in Enzymology, Academic Press, Inc.; Gene Transfer Vectors for Mammalian Cells, J. H. Miller and M. P. Calos eds. (Cold Spring Harbor Laboratory, 1987); Methods in Enzymology, Vol. 154 and 155, Wu and Grossman, eds., and Wu, ed., respectively (Academic Press, 1987), Immunochemical Methods in Cell and Molecular Biology, R. J. Mayer and J. H. Walker, eds. (Academic Press London, Harcourt Brace U.S., 1987), Protein Purification: Principles and Practice, 2nd ed. (Springer-Verlag, N.Y. (1987), and Handbook of Experimental Immunology, Vol. I-IV, D. M. Weir et al., (Blackwell Scientific Publications, 1986); Kitts et al., Biotechniques 14:810-817 (1993); Munemitsu et al., Mol. and Cell. Biol. 10:5977-5982 (1990).

Thus, the present invention contemplates the genetic modification of cells (e.g., immortalized cells such as NIKS cells or stem cells) that are used to make engineered tissues (e.g., skin equivalents). In some embodiments, the cells are modified to express an exogenous trehalose transport protein. In other embodiments, the cells are modified to express an exogenous plant late embryogenesis abundant (LEA) protein. In still other embodiments, the cells are modified to express a trehalose synthesis pathway. The present invention also contemplates combinations of the foregoing modification (e.g., modification of cells to express both an exogenous trehalose transport protein and an exogenous LEA protein).

In particularly preferred embodiments, the present contemplates cells (e.g., NIKS cells) modified to express trehalose transport protein, trehalose synthesis pathway protein, or LEA proteins, and compositions and methods for making cells (e.g., NIKS cells) expressing trehalose transport protein, trehalose synthesis pathway protein, or LEA proteins. In preferred embodiments, the cells are induced to express trehalose transport protein, trehalose synthesis pathway protein, or LEA proteins through transfection with an expression vector containing DNA encoding trehalose transport protein, trehalose synthesis pathway protein, or LEA proteins. An expression vector containing DNA encoding trehalose transport protein, trehalose synthesis pathway protein, or LEA proteins can be produced by operably linking the DNA to one or more regulatory sequences such that the resulting vector is operable in a desired host (e.g., a NIKS cell).

In some embodiments of the present invention, the full length trehalose transport protein, trehalose synthesis pathway protein, or LEA proteins or fragment thereof is expressed as a fusion protein by linking, in the correct frame and orientation, the 5′ end of the appropriate cDNA to the coding sequence of another molecule that facilitates either intracellular or extracellular production of the polypeptide.

In certain embodiments, a cDNA encoding trehalose transport protein, trehalose synthesis pathway protein, or LEA proteins is cloned into a cloning vector. In preferred embodiments, a TA cloning kit may be employed to facilitate this process.

A regulatory sequence that can be linked to DNA encoding trehalose transport protein, trehalose synthesis pathway protein, or LEA proteins in an expression vector is a promoter that is operable in the host cell in which the protein is to be expressed. Optionally, other regulatory sequences can be used herein, such as one or more of an enhancer sequence, an intron with functional splice donor and acceptance sites, a signal sequence for directing secretion of the protein, a polyadenylation sequence, other transcription terminator sequences, and a sequence homologous to the host cell genome. Other sequences, such as origin of replication, can be added to the vector as well to optimize expression of the desired protein. Further, a selectable marker can be present in the expression vector for selection of the presence thereof in the transformed host cells.

Any promoter that would allow expression of the trehalose transport protein, trehalose synthesis pathway proteins, or LEA protein in a desired host can be used in the present invention. Mammalian promoter sequences that can be used herein are those from mammalian viruses that are highly expressed and that have a broad host range. Examples include the SV40 early promoter, the Cytomegalovirus (“CMV”) immediate early promoter mouse mammary tumor virus long terminal repeat (“LTR”) promoter, adenovirus major late promoter (Ad MLP), and Herpes Simplex Virus (“HSV”) promoter. In addition, promoter sequences derived from non-viral genes, such as the murine metallothionein gene, are also useful herein. These promoters can further be either constitutive or regulated, such as those that can be induced with glucocorticoids in hormone-responsive cells. In preferred embodiments, trehalose transport protein DNA is operably linked to the promoter in pTETon plasmid (Clontech) and transfected into the target cells (e.g., NIKS cells).

The present invention is not limited to the use of any particular homolog or variant of trehalose transport protein, trehalose synthesis pathway protein, or LEA proteins. Indeed, a variety of trehalose transport protein, trehalose synthesis pathway protein, or LEA proteins variants may be used so long as they retain at least some of the activity of the corresponding wild-type protein. In particular, it is contemplated that proteins encoded by SEQ ID NOs: 1, 3, 4, 5, 6, and 7 find use in the present invention. Additionally, it is contemplated that variants encoded by sequences that hybridize to SEQ ID NOs: 1, 3, 4, 5, 6, and 7 under conditions ranging from low to high stringency will find use in the present invention. Functional variants can be screened for by expressing the variant in an appropriate vector (described in more detail below) in keratinocytes, using the keratinocytes to produce a skin equivalent, and analyzing the skin equivalent for gene expression.

In some embodiments, variants result from mutation, (i.e., a change in the nucleic acid sequence) and generally produce altered mRNAs or polypeptides whose structure or function may or may not be altered. Any given gene may have none, one, or many variant forms. Common mutational changes that give rise to variants are generally ascribed to deletions, additions or substitutions of nucleic acids. Each of these types of changes may occur alone, or in combination with the others, and at the rate of one or more times in a given sequence.

It is contemplated that it is possible to modify the structure of a polypeptide having a function (e.g., trehalose transport protein, LEA, or trehalose synthesis pathway gene function) for such purposes such as increasing binding affinity of the protein for its ligand. Such modified polypeptides or nucleic acids are considered functional equivalents of peptides having an activity of the protein, as defined herein. A modified peptide can be produced in which the nucleotide sequence encoding the polypeptide has been altered, such as by substitution, deletion, or addition. In particularly preferred embodiments, these modifications do not significantly reduce the activity of the modified protein. In other words, construct “X” can be evaluated in order to determine whether it is a member of the genus of modified or variant proteins of the present invention as defined functionally, rather than structurally. In preferred embodiments, the activity of variant or mutant protein is evaluated by the methods described herein.

Moreover, as described above, variant forms of trehalose transport protein, trehalose synthesis pathway protein, or LEA proteins are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, pg. 17-21, 2nd ed, WH Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

B) Modified Cell Lines

The present invention is not limited to the modification of any particular cell line. Indeed the present invention contemplates the modification of a variety of cell lines so that they can be efficiently preserved by freezing and/or drying and subsequently used for therapeutic or other purposes.

In some preferred embodiments, the cells lines that are modified with trehalose transport protein, trehalose synthesis pathway protein, or LEA proteins, or combinations thereof include stem cell lines. Stem cells may be derived from two sources, differentiated cells and embryos. For example, U.S. Pat. No. 5,843,780 to Thompson describes the production of stem cell lines from human embryos. Examples of adult stem cells include hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

Primate embryonic stem cells may be preferably obtained by the methods disclosed in U.S. Pat. Nos. 5,843,780 and 6,200,806, each of which is incorporated herein by reference. Primate (including human) stem cells may also be obtained from commercial sources such as WiCell, Madison, Wis. A preferable medium for isolation of embryonic stem cells is “ES medium.” ES medium consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Preferably, fetal bovine serum batches are compared by testing clonal plating efficiency of a low passage mouse ES cell line (ESⱼₜ₃), a cell line developed just for the purpose of this test. FBS batches must be compared because it has been found that batches vary dramatically in their ability to support embryonic cell growth, but any other method of assaying the competence of FBS batches for support of embryonic cells will work as an alternative.

Primate ES cells are isolated on a confluent layer of murine embryonic fibroblast in the presence of ES cell medium. Embryonic fibroblasts are preferably obtained from 12 day old fetuses from outbred CF1 mice (SASCO), but other strains may be used as an alternative. Tissue culture dishes are preferably treated with 0.1% gelatin (type I; Sigma).

For rhesus monkey embryos, adult female rhesus monkeys (greater than four years old) demonstrating normal ovarian cycles are observed daily for evidence of menstrual bleeding (day 1 of cycle=the day of onset of menses). Blood samples are drawn daily during the follicular phase starting from day 8 of the menstrual cycle, and serum concentrations of lutenizing hormone are determined by radioimmunoassay. The female is paired with a male rhesus monkey of proven fertility from day 9 of the menstrual cycle until 48 hours after the lutenizing hormone surge; ovulation is taken as the day following the leutenizing hormone surge. Expanded blastocysts are collected by non-surgical uterine flushing at six days after ovulation. This procedure routinely results in the recovery of an average 0.4 to 0.6 viable embryos per rhesus monkey per month, Seshagiri et al. Am J Primatol 29:81-91, 1993.

For marmoset embryos, adult female marmosets (greater than two years of age) demonstrating regular ovarian cycles are maintained in family groups, with a fertile male and up to five progeny. Ovarian cycles are controlled by intramuscular injection of 0.75 g of the prostaglandin PGF2a analog cloprostenol (Estrumate, Mobay Corp, Shawnee, Kans.) during the middle to late luteal phase. Blood samples are drawn on day 0 (immediately before cloprostenol injection), and on days 3, 7, 9, 11, and 13. Plasma progesterone concentrations are determined by ELISA. The day of ovulation is taken as the day preceding a plasma progesterone concentration of 10 ng/ml or more. At eight days after ovulation, expanded blastocysts are recovered by a non-surgical uterine flush procedure, Thomson et al. “Non-surgical uterine stage preimplantation embryo collection from the common marmoset,” J Med Primatol, 23:333-336 (1994). This procedure results in the average production of 1.0 viable embryos per marmoset per month.

The zona pellucida is removed from blastocysts by brief exposure to pronase (Sigma). For immunosurgery, blastocysts are exposed to a 1:50 dilution of rabbit anti-marmoset spleen cell antiserum (for marmoset blastocysts) or a 1:50 dilution of rabbit anti-rhesus monkey (for rhesus monkey blastocysts) in DMEM for 30 minutes, then washed for 5 minutes three times in DMEM, then exposed to a 1:5 dilution of Guinea pig complement (Gibco) for 3 minutes.

After two further washes in DMEM, lysed trophectoderm cells are removed from the intact inner cell mass (ICM) by gentle pipetting, and the ICM plated on mouse inactivated (3000 rads gamma irradiation) embryonic fibroblasts. After 7-21 days, ICM-derived masses are removed from endoderm outgrowths with a micropipette with direct observation under a stereo microscope, exposed to 0.05% Trypsin-EDTA (Gibco) supplemented with 1% chicken serum for 3-5 minutes and gently dissociated by gentle pipetting through a flame polished micropipette.

Dissociated cells are replated on embryonic feeder layers in fresh ES medium, and observed for colony formation. Colonies demonstrating ES-like morphology are individually selected, and split again as described above. The ES-like morphology is defined as compact colonies having a high nucleus to cytoplasm ratio and prominent nucleoli. Resulting ES cells are then routinely split by brief trypsinization or exposure to Dulbecco's Phosphate Buffered Saline (without calcium or magnesium and with 2 mM EDTA) every 1-2 weeks as the cultures become dense. Early passage cells are also frozen and stored in liquid nitrogen.

The methods of the present invention are not limited to the use of primate embryonic stem cells. Indeed, the use of embryonic stem cells from other species are contemplated, including, but not limited to mice, rats, pigs, cattle and sheep. Methods for obtaining pluripotent cells from these species have been previously described. See, e.g., U.S. Pat. Nos. 5,453,357; 5,523,226; 5,589,376; 5,340,740; and 5,166,065 (all of which are specifically incorporated herein by reference); as well as, Evans, et al., Theriogenology 33(1):125-128, 1990; Evans, et al., Theriogenology 33(1):125-128, 1990; Notarianni, et al., J. Reprod. Fertil. 41(Suppl.):51-56, 1990; Giles, et al., Mol. Reprod. Dev. 36:130-138, 1993; Graves, et al., Mol. Reprod. Dev. 36:424-433, 1993; Sukoyan, et al., Mol. Reprod. Dev. 33:418-431, 1992; Sukoyan, et al., Mol. Reprod. Dev. 36:148-158, 1993; Iannaccone, et al., Dev. Biol. 163:288-292, 1994; Evans & Kaufman, Nature 292:154-156, 1981; Martin, Proc Natl Acad Sci USA 78:7634-7638, 1981; Doetschman et al. Dev Biol 127:224-227, 1988); Giles et al. Mol Reprod Dev 36:130-138, 1993; Graves & Moreadith, Mol Reprod Dev 36:424-433, 1993 and Bradley, et al., Nature 309:255-256, 1984.

The present invention also contemplates the use of non-embryonic stem cells. Mesenchymal stem cells (MSCs) can be derived from marrow, periosteum, dermis and other tissues of mesodermal origin (See, e.g., U.S. Pat. Nos. 5,591,625 and 5,486,359, each of which is incorporated herein by reference). MSCs are the formative pluripotential blast cells that differentiate into the specific types of connective tissues (i.e. the tissues of the body that support the specialized elements; particularly adipose, areolar, osseous, cartilaginous, elastic, marrow stroma, muscle, and fibrous connective tissues) depending upon various in vivo or in vitro environmental influences. Although these cells are normally present at very low frequencies in bone marrow, various methods have been described for isolating, purifying, and greatly replicating the marrow-derived mesenchymal stems cells in culture, i.e. in vitro (See also U.S. Pat. Nos. 5,197,985 and 5,226,914 and PCT Publication No. WO 92/22584, each of which are incorporated herein by reference).

Various methods have also been described for the isolation of hematopoietic stem cells (See, e.g., U.S. Pat. Nos. 5,061,620; 5,750,397; 5,716,827 all of which are incorporated herein by reference). It is contemplated that the methods of the present invention can be used to produce lymphoid, myeloid and erythroid cells from hematopoietic stem cells. The lymphoid lineage, comprising B-cells and T-cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies in the blood stream, provides protection against neoplastic cells, scavenges foreign materials in the blood stream, produces platelets, and the like. The erythroid lineage provides the red blood cells, which act as oxygen carriers.

The present invention also contemplates the use of neural stem cells, which are generally isolated from developing fetuses. The isolation, culture, and use of neural stem cells are described in U.S. Pat. Nos. 5,654,183; 5,672,499; 5,750,376; 5,849,553; and 5,968,829, all of which are incorporated herein by reference. It is contemplated that the methods of the present invention can use neural stem cells to produce neurons, glia, melanocytes, cartilage and connective tissue of the head and neck, stroma of various secretory glands and cells in the outflow tract of the heart.

In some other preferred embodiments, the cells lines that are modified with trehalose transport protein, trehalose synthesis pathway protein, or LEA proteins, or combinations thereof include any source of cells or cell line that can stratify into squamous epithelia. Accordingly, the present invention is not limited to the use of any particular source of cells that are capable of differentiating into squamous epithelia. Indeed, the present invention contemplates the use of a variety of cell lines and sources that can differentiate into squamous epithelia, including both primary and immortalized keratinocytes. Sources of cells include keratinocytes and dermal fibroblasts biopsied from humans and cavaderic donors (Auger et al., In Vitro Cell. Dev. Biol.—Animal 36:96-103; U.S. Pat. Nos. 5,968,546 and 5,693,332, each of which is incorporated herein by reference), neonatal foreskins (Asbill et al., Pharm. Research 17(9): 1092-97 (2000); Meana et al., Burns 24:621-30 (1998); U.S. Pat. Nos. 4,485,096; 6,039,760; and 5,536,656, each of which is incorporated herein by reference), and immortalized keratinocytes cell lines such as NM1 cells (Baden, In Vitro Cell. Dev. Biol. 23(3):205-213 (1987)), HaCaT cells (Boucamp et al., J. cell. Boil. 106:761-771 (1988)); and NIKS cells (Cell line BC-1-Ep/SL; U.S. Pat. No. 5,989,837, incorporated herein by reference; ATCC CRL-12191). Each of these cell lines can be cultured or genetically modified as described below in order to produce a cell line capable of expressing or co-expressing the desired protein(s).

In particularly preferred embodiments, NIKS cells are utilized. The discovery of a novel human keratinocyte cell line (near-diploid immortalized keratinocytes or NIKS) provides an opportunity to genetically engineer human keratinocytes for new in vitro testing methods. A unique advantage of the NIKS cells is that they are a consistent source of genetically-uniform, pathogen-free human keratinocytes. For this reason, they are useful for the application of genetic engineering and genomic gene expression approaches to provide skin equivalent cultures with properties more similar to human skin. Such systems will provide an important alternative to the use of animals for testing compounds and formulations. The NIKS keratinocyte cell line, identified and characterized at the University of Wisconsin, is nontumorigenic, exhibits a stable karyotype, and exhibits normal differentiation both in monolayer and organotypic culture. NIKS cells form fully stratified skin equivalents in culture. These cultures are indistinguishable by all criteria tested thus far from organotypic cultures formed from primary human keratinocytes. Unlike primary cells however, the immortalized NIKS cells will continue to proliferate in monolayer culture indefinitely. This provides an opportunity to genetically manipulate the cells and isolate new clones of cells with new useful properties (Allen-Hoffmann et al., J. Invest. Dermatol., 114(3): 444-455 (2000)).

The NIKS cells arose from the BC-1-Ep strain of human neonatal foreskin keratinocytes isolated from an apparently normal male infant. In early passages, the BC-1-Ep cells exhibited no morphological or growth characteristics that were atypical for cultured normal human keratinocytes. Cultivated BC-1-Ep cells exhibited stratification as well as features of programmed cell death. To determine replicative lifespan, the BC-1-Ep cells were serially cultivated to senescence in standard keratinocyte growth medium at a density of 3×10⁵ cells per 100-mm dish and passaged at weekly intervals (approximately a 1:25 split). By passage 15, most keratinocytes in the population appeared senescent as judged by the presence of numerous abortive colonies which exhibited large, flat cells. However, at passage 16, keratinocytes exhibiting a small cell size were evident. By passage 17, only the small-sized keratinocytes were present in the culture and no large, senescent keratinocytes were evident. The resulting population of small keratinocytes that survived this putative crisis period appeared morphologically uniform and produced colonies of keratinocytes exhibiting typical keratinocyte characteristics including cell-cell adhesion and apparent squame production. The keratinocytes that survived senescence were serially cultivated at a density of 3×10⁵ cells per 100-mm dish. Typically the cultures reached a cell density of approximately 8×10⁶ cells within 7 days. This stable rate of cell growth was maintained through at least 59 passages, demonstrating that the cells had achieved immortality. The keratinocytes that emerged from the original senescencing population were originally designated BC-1-Ep/Spontaneous Line and are now termed NIKS. The NIKS cell line has been screened for the presence of proviral DNA sequences for HIV-1, HIV-2, EBV, CMV, HTLV-1, HTLV-2, HBV, HCV, B-19 parvovirus, HPV-16 and HPV-31 using either PCR or Southern analysis. None of these viruses were detected.

Chromosomal analysis was performed on the parental BC-1-Ep cells at passage 3 and NIKS cells at passages 31 and 54. The parental BC-1-Ep cells have a normal chromosomal complement of 46, XY. At passage 31, all NIKS cells contained 47 chromosomes with an extra isochromosome of the long arm of chromosome 8. No other gross chromosomal abnormalities or marker chromosomes were detected. At passage 54, all cells contained the isochromosome 8.

The DNA fingerprints for the NIKS cell line and the BC-1-Ep keratinocytes are identical at all twelve loci analyzed demonstrating that the NIKS cells arose from the parental BC-1-Ep population. The odds of the NIKS cell line having the parental BC-1-Ep DNA fingerprint by random chance is 4×10⁻¹⁶. The DNA fingerprints from three different sources of human keratinocytes, ED-1-Ep, SCC4 and SCC13y are different from the BC-1-Ep pattern. This data also shows that keratinocytes isolated from other humans, ED-1-Ep, SCC4, and SCC13y, are unrelated to the BC-1-Ep cells or each other. The NIKS DNA fingerprint data provides an unequivocal way to identify the NIKS cell line.

Loss of p53 function is associated with an enhanced proliferative potential and increased frequency of immortality in cultured cells. The sequence of p53 in the NIKS cells is identical to published p53 sequences (GenBank accession number: M14695). In humans, p53 exists in two predominant polymorphic forms distinguished by the amino acid at codon 72. Both alleles of p53 in the NIKS cells are wild-type and have the sequence CGC at codon 72, which codes for an arginine. The other common form of p53 has a proline at this position. The entire sequence of p53 in the NIKS cells is identical to the BC-1-Ep progenitor cells. Rb was also found to be wild-type in NIKS cells.

Anchorage-independent growth is highly correlated to tumorigenicity in vivo. For this reason, the anchorage-independent growth characteristics of NIKS cells in agar or methylcellulose-containing medium was investigated. After 4 weeks in either agar- or methylcellulose-containing medium, NIKS cells remained as single cells. The assays were continued for a total of 8 weeks to detect slow growing variants of the NIKS cells. None were observed.

To determine the tumorigenicity of the parental BC-1-Ep keratinocytes and the immortal NIKS keratinocyte cell line, cells were injected into the flanks of athymic nude mice. The human squamous cell carcinoma cell line, SCC4, was used as a positive control for tumor production in these animals. The injection of samples was designed such that animals received SCC4 cells in one flank and either the parental BC-1-Ep keratinocytes or the NIKS cells in the opposite flank. This injection strategy eliminated animal to animal variation in tumor production and confirmed that the mice would support vigorous growth of tumorigenic cells. Neither the parental BC-1-Ep keratinocytes (passage 6) nor the NIKS keratinocytes (passage 35) produced tumors in athymic nude mice.

NIKS cells were analyzed for the ability to undergo differentiation in both surface culture and organotypic culture. For cells in surface culture, a marker of squamous differentiation, the formation cornified envelopes was monitored. In cultured human keratinocytes, early stages of cornified envelope assembly result in the formation of an immature structure composed of involucrin, cystatin-α and other proteins, which represent the innermost third of the mature cornified envelope. Less than 2% of the keratinocytes from the adherent BC-1-Ep cells or the NIKS cell line produce cornified envelopes. This finding is consistent with previous studies demonstrating that actively growing, subconfluent keratinocytes produce less than 5% cornified envelopes. To determine whether the NIKS cell line is capable of producing cornified envelopes when induced to differentiate, the cells were removed from surface culture and suspended for 24 hours in medium made semi-solid with methylcellulose. Many aspects of terminal differentiation, including differential expression of keratins and cornified envelope formation can be triggered in vitro by loss of keratinocyte cell-cell and cell-substratum adhesion. The NIKS keratinocytes produced as many as and usually more cornified envelopes than the parental keratinocytes. These findings demonstrate that the NIKS keratinocytes are not defective in their ability to initiate the formation of this cell type-specific differentiation structure.

To confirm that the NIKS keratinocytes can undergo squamous differentiation, the cells were cultivated in organotypic culture. Keratinocyte cultures grown on plastic substrata and submerged in medium replicate but exhibit limited differentiation. Specifically, human keratinocytes become confluent and undergo limited stratification producing a sheet consisting of 3 or more layers of keratinocytes. By light and electron microscopy there are striking differences between the architecture of the multilayered sheets formed in tissue culture and intact human skin. In contrast, organotypic culturing techniques allow for keratinocyte growth and differentiation under in vivo-like conditions. Specifically, the cells adhere to a physiological substratum consisting of dermal fibroblasts embedded within a fibrillar collagen base. The organotypic culture is maintained at the air-medium interface. In this way, cells in the upper sheets are air-exposed while the proliferating basal cells remain closest to the gradient of nutrients provided by diffusion through the collagen gel. Under these conditions, correct tissue architecture is formed. Several characteristics of a normal differentiating epidermis are evident. In both the parental cells and the NIKS cell line a single layer of cuboidal basal cells rests at the junction of the epidermis and the dermal equivalent. The rounded morphology and high nuclear to cytoplasmic ratio is indicative of an actively dividing population of keratinocytes. In normal human epidermis, as the basal cells divide they give rise to daughter cells that migrate upwards into the differentiating layers of the tissue. The daughter cells increase in size and become flattened and squamous. Eventually these cells enucleate and form cornified, keratinized structures. This normal differentiation process is evident in the upper layers of both the parental cells and the NIKS cells. The appearance of flattened squamous cells is evident in the upper layers of keratinocytes and demonstrates that stratification has occurred in the organotypic cultures. In the uppermost part of the organotypic cultures the enucleated squames peel off the top of the culture. To date, no histological differences in differentiation at the light microscope level between the parental keratinocytes and the NIKS keratinocyte cell line grown in organotypic culture have been observed.

To observe more detailed characteristics of the parental (passage 5) and NIKS (passage 38) organotypic cultures and to confirm the histological observations, samples were analyzed using electron microscopy. Parental cells and the immortalized human keratinocyte cell line, NIKS, were harvested after 15 days in organotypic culture and sectioned perpendicular to the basal layer to show the extent of stratification. Both the parental cells and the NIKS cell line undergo extensive stratification in organotypic culture and form structures that are characteristic of normal human epidermis. Abundant desmosomes are formed in organotypic cultures of parental cells and the NIKS cell line. The formation of a basal lamina and associated hemidesmosomes in the basal keratinocyte layers of both the parental cells and the cell line was also noted.

Hemidesmosomes are specialized structures that increase adhesion of the keratinocytes to the basal lamina and help maintain the integrity and strength of the tissue. The presence of these structures was especially evident in areas where the parental cells or the NIKS cells had attached directly to the porous support. These findings are consistent with earlier ultrastructural findings using human foreskin keratinocytes cultured on a fibroblast-containing porous support. Analysis at both the light and electron microscopic levels demonstrate that the NIKS cell line in organotypic culture can stratify, differentiate, and form structures such as desmosomes, basal lamina, and hemidesmosomes found in normal human epidermis.

C) Production of Organs and Tissues

In some embodiments of the invention, the genetically modified cells described above are used to produce organs and tissues. In some preferred embodiments, the cells (e.g., modified NIKS cells) are used to produce human skin equivalents. The production of human skin equivalents from NIKS cells is described in U.S. Pat. No. 5,989,837 (which is incorporated herein by reference) and in the examples.

The modified cells may also be used to produce other types of organs and tissues. The in vitro growth of organs is described in U.S. Pat. No. 6,140,039 (tendons and ligaments); U.S. Pat. No. 5,902,741 (cartilage); U.S. Pat. No. 5,849,588 (liver); U.S. Pat. No. 6,022,743 (pancreas); U.S. Pat. No. 5,516,680 (kidney); U.S. Pat. No. 5,266,480 (skin); U.S. Pat. Nos. 6,121,042; 5,962,325; 5,510,254; 5,518,915; 5,843,766; 5,863,531; 5,763,267; 5,785,964; 5,591,625; 5,486,359 and 5,827,729 all of which are incorporated herein by reference.

D) Preservation of Organs and Tissues

In some preferred embodiments, the modified cells or organs or tissues comprising the modified cells are preserved by freezing and/or drying. It is contemplated that the techniques of freezing and/or drying provide an extended shelf-life for the modified cells and organs and tissues comprising the modified cells. In preferred embodiments, the frozen and/or dried cells, tissues and organs comprising modified cells have a shelf-life of greater than about one week at ambient temperatures (e.g., temperatures ranging from about 0° C. to about 38° C.). In more preferred embodiments, the frozen and/or dried cells, tissues and organs comprising modified cells have a shelf-life of greater than about one month at ambient temperatures. In the most preferred embodiments, the frozen and/or dried cells, tissues and organs comprising modified cells have a shelf-life of greater than about six months at ambient temperatures. Likewise, in preferred embodiments, frozen cells, tissues and organs comprising modified cells have a shelf-life of greater than about one week at freezing temperature (e.g., temperatures ranging from about −180° C. to about 0° C.). In more preferred embodiments, the frozen cells, tissues and organs comprising modified cells have a shelf-life of greater than about one month at freezing temperatures. In the most preferred embodiments, the frozen cells, tissues and organs comprising modified cells have a shelf-life of greater than about six months at freezing temperatures.

In still other preferred embodiments, the present invention provides frozen and/or dried modified cells and tissues and organs comprising modified cells that that exhibit greater than about 70% viability after thawing and/or rehydration. In more preferred embodiments, the present invention provides frozen and/or dried modified cells and tissues and organs comprising modified cells that that exhibit greater than about 80% viability after thawing and/or rehydration. In the most preferred embodiments, the present invention provides frozen and/or dried modified cells and tissues and organs comprising modified cells that that exhibit greater than about 90% viability after thawing and/or rehydration.

In some preferred embodiments, the cells, organs or tissues are vitrified. In further preferred embodiments, the cells, organs, or tissues are freeze-dried (i.e., the water in the cells, organs, or tissues is removed while the cells, organs, or tissues are in the frozen state). In other preferred embodiments, the cells, organs, or tissues are air-dried. In some particularly preferred embodiments, the cells, organs, or tissues containing trehalose are frozen in the presence of trehalose and an oxyanion. The present invention is not limited to the use of any particular oxyanion. Indeed, the use of a variety of oxyanions is contemplated, including, but not limited to borate, phosphate, carbonate, sulfate and nitrate.

E) Therapeutic Uses

It is contemplated that the preserved cells, organs, and tissues of the present invention may be used therapeutically.

In some embodiments, the cells, organs, and tissues are utilized to treat chronic skin wounds. Successful treatment of chronic skin wounds (e.g., venous ulcers, diabetic ulcers, pressure ulcers) is a serious problem. The healing of such a wound often times takes well over a year of treatment. Treatment options currently include dressings and debridement (use of chemicals or surgery to clear away necrotic tissue), and/or antibiotics in the case of infection. These treatment options take extended periods of time and high amounts of patient compliance. As such, a therapy than can increase a practitioner's success in healing chronic wounds and accelerate the rate of wound healing would meet an unmet need in the field. Accordingly, the present invention contemplates treatment of skin wounds with skin equivalents comprising the modified cells of the present invention (e.g., modified NIKS cells). In some embodiments, modified NIKS cells are topically applied to wound sites. In other embodiments, skin equivalents comprising modified NIKS cells are used for engraftment on partial thickness wounds. In other embodiments, skin equivalents comprising modified NIKS cells are used for engraftment on full thickness wounds. In other embodiments, skin equivalents comprising modified NIKS cells are used to treat numerous types of internal wounds, including, but not limited to, internal wounds of the mucous membranes that line the gastrointestinal tract, ulcerative colitis, and inflammation of mucous membranes that may be caused by cancer therapies. In still other embodiments, skin equivalents comprising modified NIKS cells expressing are used as a temporary or permanent wound dressing.

Skin equivalents comprising modified cells also find use in wound closure and burn treatment applications. The use of autografts and allografts for the treatment of burns and wound closure is described in Myers et al., A. J. Surg. 170(1):75-83 (1995) and U.S. Pat. Nos. 5,693,332; 5,658,331; and 6,039,760, each of which is incorporated herein by reference. In some embodiments, the skin equivalents may be used in conjunction with dermal replacements such as DERMAGRAFT or EXPRESSGRAFT. In other embodiments, the skin equivalents are produced using both a standard source of keratinocytes (e.g., NIKS cells) and keratinocytes from the patient that will receive the graft. Therefore, the skin equivalent contains keratinocytes from two different sources. In still further embodiments, the skin equivalent contains keratinocytes from a human tissue isolate. Accordingly, the present invention provides methods for wound closure, including wounds caused by burns, comprising providing a skin equivalent and a patient suffering from a wound and treating the patient with the skin equivalent under conditions such that the wound is closed.

In still further embodiments, the modified cells are engineered to provide additional therapeutic agents to a subject. The present invention is not limited to the delivery of any particular therapeutic agent. Indeed, it is contemplated that a variety of therapeutic agents may be delivered to the subject, including, but not limited to, enzymes, peptides, peptide hormones, other proteins, ribosomal RNA, ribozymes, and antisense RNA. These therapeutic agents may be delivered for a variety of purposes, including but not limited to the purpose of correcting genetic defects. In some particular preferred embodiments, the therapeutic agent is delivered for the purpose of detoxifying a patient with an inherited inborn error of metabolism (e.g., aminoacidopathesis) in which the graft serves as wild-type tissue. It is contemplated that delivery of the therapeutic agent corrects the defect. In some embodiments, the modified cells are co-transformed with a DNA construct encoding a therapeutic agent (e.g., insulin, clotting factor IX, erythropoietin, etc) and the cells grafted onto the subject. The therapeutic agent is then delivered to the patient's bloodstream or other tissues from the graft. In preferred embodiments, the nucleic acid encoding the therapeutic agent is operably linked to a suitable promoter. The present invention is not limited to the use of any particular promoter. Indeed, the use of a variety of promoters is contemplated, including, but not limited to, inducible, constitutive, tissue specific, and keratinocyte specific promoters. In some embodiments, the nucleic acid encoding the therapeutic agent is introduced directly into the keratinocytes (i.e., by calcium phosphate co-precipitation or via liposome transfection). In other preferred embodiments, the nucleic acid encoding the therapeutic agent is provided as a vector and the vector is introduced into the keratinocytes by methods known in the art. In some embodiments, the vector is an episomal vector such as a plasmid. In other embodiments, the vector integrates into the genome of the keratinocytes. Examples of integrating vectors include, but are not limited to, retroviral vectors, adeno-associated virus vectors, and transposon vectors.

It is further contemplated that the cell lines described above find use in a variety of cell transplant therapies. In particular, the cell lines described above can be differentiated into any desired cell type. In some embodiments, hematopoietic cell lines are generated from the cell lines described above and used to treat diseases that require bone marrow transplantation such as ovarian cancer and leukemia, as well as diseases that attack the immune system such as AIDS. In still other embodiments, the cell lines described above are used to generate neural cell lines. Diseases treatable by transplantation of such cell lines include Parkinson's disease, Alzheimer's disease, ALS, and cerebral palsy. Other diseases treatable by cell transplant therapy include spinal cord injuries, multiple sclerosis, muscular dystrophy, diabetes, liver diseases, heart diseases, cartilage replacement, burns, foot ulcers, and kidney diseases.

Accordingly, the present invention provides methods for transplant therapy comprising providing a modified cell line as described above and a subject, and transplanting the cell line into the subject under conditions such that said cell line produces progeny cells having a particular phenotype. For example, in some embodiments, the cell line is transplanted into the nervous system of a subject (e.g., brain or spinal cord) and the progeny cells adopt a neural cell phenotype. In other embodiments, the cell lines are transplanted into the liver of the subject and the progeny of the transplanted cells display a mesodermal cell phenotype.

The present invention also provides methods for cell transplant therapy comprising providing a subject and a modified cell line or cell line, and transplanting the cell line into the subject under conditions such that the cell line differentiated into a particular fate or contributes to a particular tissue. In some embodiments, the most primitive form of the modified cells are utilized in the cell transplant therapy (i.e, cells having a stem-cell morphology and expressing embryonic stem cell specific markers). In other embodiments, the modified cell lines are induced to differentiate into a particular fate in vitro (i.e., a hematopoietic stem cell or neural stem cell) and then transplanted. In still further embodiments, the modified cells are transplanted into SCID mice and allowed to differentiate into a variety of cell types. The desired cell type is then isolated from the SCID mouse, expanded in vitro, and used in the cell transplant therapy.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); mol (nanomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); U (units), mU (milliunits); min. (minutes); sec. (seconds); % (percent); kb (kilobase); bp (base pair); PCR (polymerase chain reaction); BSA (bovine serum albumin).

### Example 1

This example describes a method for the production of skin equivalents.

Media. The organotypic culture process uses six different culture media: 3T3 feeder cell medium (TM); human fibroblast growth medium (FGM); NIKS medium (NM); plating medium (PM); stratification medium A (SMA); and stratification medium B (SMB). TM is used to propagate 3T3 cells that act as feeder cells for NIKS cells in monolayer culture. TM is a mixture of Dulbecco's modified Eagle's medium (DME, GibcoBRL) supplemented with 10% calf serum (Hyclone). FGM is a commercially available fibroblast growth medium (Clonetics) that is used to propagate the normal human dermal fibroblast cells (NHDFs) for use in STRATAGRAFT skin equivalent and STATATEST skin equivalent dermal equivalent layers. NM is used to grow NIKS keratinocytes. NM is a 3:1 mixture of Ham's F-12 medium (GibcoBRL) and DME supplemented with 2.5% fetal clone II (Hyclone), 0.4 μg/ml hydrocortisone (Calbiochem), 8.4 ng/ml cholera toxin (ICN), 5 μg/ml insulin (Sigma), 24 μg/ml adenine (Sigma) and 10 ng/ml epidermal growth factor (EGF, R&D systems). PM is the medium used when NIKS cells are seeded onto a dermal equivalent. PM is the same NM with the exception that EGF is removed and CaCl₂ (Sigma) is supplemented to a final calcium concentration of 1.88 nm. SMA is the same as PM with the addition of 1 mg/ml bovine serum albumin (BSA), 1 μM isoproterenol, 10 μM carnitine, 10 μM serine, 25 μM oleic acid, 15 μM linoleic acid, 7 μM arachidonic acid, 1 μM α-tocopherol, 0.05 mg/ml ascorbic acid (all from Sigma), and 1 ng/ml EGF. SMB is used during the epidermal stratification phase of STRATATEST skin equivalent and STRATAGRAFT skin equivalent growth. SMB is the same as SMA but without the presence of the fetal clone II serum supplement.

Feeder preparation. Prior to starting STRATAGRAFT skin equivalent organotypic cultures, 3T3 feeder cells are prepared and then used either fresh or frozen for later use. 3T3 cells are grown to confluence and treated with mitomycin-C (100 μl mitomycin-C in 5 ml of TM, Roche) for two hours. The cells are then washed, resuspended, and plated at a density of 1.25×1 per 100 mm tissue culture dish to support NIKS growth. If frozen feeders are used, single frozen ampoule containing 1 ml with 2.5×10⁶ is thawed, diluted with fresh TM and plated onto a single 100 mm tissue culture dish. This is done for as many dishes as will be needed for NIKS cell growth one prior to plating the NIKS cells.

Dermal equivalent preparation. On day 0, frozen NHDF cells are thawed and plated. The cells are fed FGM the next day (day 1) to residual cryoprotectant and again on day 3. On day 4, they are harvested for in the dermal equivalent. To prepare the dermal equivalent, tat-tail collagen (Type I, Becton-Dickinson) is first diluted to 3 mg/ml in 0.03N acetic acid and chilled on ice. A mixture of concentrated Ham's F12 medium (8.7× normal strength and buffered with HEPES at pH 7.5) is mixed with fetal clone II (supplemented bovine serum). These two solutions are 11.5 and 10% of the final solution volume. 1N NaOH is added to the medium mixture (2.5% of final solution). The diluted collagen is then added (74%) to the mixture. A 2% volume of suspended fibroblasts (1×10⁶ for STRATAGRAFT skin equivalent) is added to the mixture. The solution is mixed gently but thoroughly and 100 μl is aliquoted into tissue culture inserts (MILLICELL from Millipore Corp.) placed 25 in a 100 mm tissue culture dish. STRATAGRAFT skin equivalent uses TRANSWELL inserts from Corning. A 13 ml dermal equivalent is poured into each insert. After 30 minutes for gel formation, the dish is flooded with 20 ml of F12 medium supplemented with 10% fetal clone II. One or two drops of the F-12-serum mix are placed on the surface of each dermal equivalent. For STRATAGRAFT skin equivalent, 80 ml of the F12-serum mix is placed around the TRANSWELL insert in a 150 mm tissue culture dish and 10 ml is placed on top of the dermal equivalent. The inserts are placed in 37° C., 5% CO₂, 90% relative humidity incubator until used. One day prior to seeding the dermal equivalents with NIKS cells, they are lifted to the air interface by placing them onto a sterile stainless steel mesh with two wicking pads (S&S Biopath) on top to supply medium through the bottom of the tissue culture insert.

NIKS Growth and Seeding. On day 0, the feeders are thawed (if necessary) and plated in TM. On day 1, NIKS cells are plated onto the feeders at a density of approximately 3×10⁵ cells per 100 mm dish. On day 2, the NIKS cells are fed fresh NM to remove residual cryoprotectant. The NIKS cells are fed again on days 4 and 6. (For STRATAGRAFT skin equivalent size cultures, the NIKS cultures are started a week earlier due to the increase in number of cells needed). On day 8, the NIKS cells are harvested, counted, and resuspended in PM. 4.65×10⁵ NIKS cells/cm² are seeded onto the surface of the MIILLICELL or TRANSWELL inserts, which have been lifted to the air interface for one day. The dishes are fed 30 ml PM (100 ml for STRATAGRAFT skin equivalent) underneath the metal lifter and placed back into the incubator. On day 10, the cultures are fed SMA. On days 12, 14, 16, 18, 20, and 22 the cultures are fed SMB. On day 12, the cultures are transferred to a 75% humidity incubator where they remain for the rest of their growth.

### Example 2

This example describes the cryopreservation of isolated NIKS cells. In this study, NIKS cells were suspended after freezing with trehalose. Roughly 500 mM trehalose represents optimal recovery though none of the trehalose samples achieved more than 50% of the glycerol-treated control. The fact that glycerol is twice as effective as extracellular trehalose emphasizes the role of osmotic damage in this process and underscores the potential benefits of intracellular trehalose and sample vitrification.

Equilibration of cells at reduced temperatures prior to cryoprocessing can improve viable cell recovery. In order to explore this effect for the NIKS cells, isolated cells were frozen at different rates in trehalose supplemented growth medium. The results indicate that longer exposure to pre-cooling improves cell recovery. This effect may be attributable to permeabilization of cells as their membranes pass from a liquid crystalline to a gel phase.

### Example 3

This example describes the construction of trehalose synthesis enzyme expression vectors. Trehalose biosynthesis requires two enzymatic activities: trehalose-6-phosphate synthase (T6PS), which catalyzes the formation of trehalose-6-phosphate from UDP-glucose and glucose-6-phosphate, and trehalose-6-phosphate phosphatase (T6PP), which generates trehalose by dephosphorylating trehalose-6-phosphate. In some embodiments, the T6PS and T6PP are encoded by the otsA and otsB genes of E. coli. Kaasen et al., Gene 145(1):9-15 (1994). In other embodiments, the otsA and otsB gene functions are one the same gene. For example, drosophila melanogaster otsA and otsB gene functions are contained on a single gene, the tps1 gene (See e.g., Chen et al., J. Biol. Chem. 277:3274 (2002) and Chen et al., J. Biol. Chem. 278:49113 (2003)). To control the timing and extent of trehalose expression, the otsA and otsB genes are expressed using the Tet-On regulatory system. Gossen et al., Proc. Natl. Acad. Sci. USA 89(12):5547-51 (1992); Gossen et al., Curr. Opin. Biotech. 5(5):516-20 (1994). This system allows for induction and termination of gene expression in the presence and absence of the tetracycline derivative doxycycline, respectively.

The otsA and otsB coding regions are isolated by PCR using primers based on published sequences (Kaasen et al., supra), E. coli genomic template DNA, and a high fidelity polymerase such as Pfu. The PCR products are cloned using the TOPO-TA cloning kit (Invitrogen). To facilitate processing, stability, and translation of otsA and otsB mRNA in the human cells, a DNA fragment containing the rabbit β-globin intron and poly(A) signal will be ligated onto the PCR products following the stop codons. The otsA and otsB coding regions are cloned into the pBI expression vector (Clontech, Palo Alto, Calif.), which contains a bi-directional promoter consisting of seven repeats of the Tet operator flanked by two minimal cytomegalovirus promoters. The integrity of the otsA and otsB coding regions is confirmed by DNA sequencing with gene-specific primers to ensure that no mutations were introduced during the PCR or cloning procedures.

### Example 4

This example describes the construction of a trehalose transport protein expression vector. The AGT1 protein of the yeast S. cerevisiae is an alpha-glucoside/H+ symporter that induces intracellular accumulation of trehalose, maltose, isomaltose, turanose, maltotriose, palatinose, and melezitose. Han et al., Mol. Microbiol. 17(6):1093-107 (1995); Plourde-Owabi et al., J. Bacteriol. 181(12):3830-2 (1999). The AGT1 coding region is amplified by PCR using primers based on published sequences. Template cDNA for this amplification is prepared by extracting RNA from yeast grown under conditions known to induce trehalose transport and converting it to cDNA with the SuperscriptII first strand cDNA synthesis kit from Invitrogen. Stambuk et al., Biochim. Biophys. Acta—General Subjects 1379(1):118-28 (1998). The AGT1 coding region is cloned using the TOPO-TA cloning kit. The AGT1 coding region is cloned into the tetracycline-responsive expression vector pTRE2-hyg (Clontech), which also contains an intron and poly(A) signal from the rabbit β-globin gene to enhance mRNA stability, processing and translation. The integrity of the coding region is confirmed by DNA sequencing using AGT-specific primers.

### Example 5

This example describes the inducible synthesis/uptake of trehalose in transiently transfected NIKS cells, MSC cells (Clonetics), and NHDF cells. Purified DNA from the vectors described in examples 3 and 4 is introduced into the appropriate cell type along with the pTet-On plasmid (Clontech). pTet-On encodes rtTA, which consists of the VP16 transactivation domain fused to the DNA binding domain of the tet repressor. The rtTA transactivator binds to the tet operator in the presence of doxycycline and induces gene expression. Cells will be transfected using TransIt-LT1 reagent (Mirus Corp., Madison, Wis.).

The transfection efficiency in the cells is optimzed by co-transfection of an easily detectable reporter gene together with the pTet-On and the expression vectors from the preceding examples. Genetic reporter systems are widely used to study eukaryotic gene expression and cellular physiology. Applications include the study of receptor activity, transcription factors, intracellular signaling, mRNA processing and protein folding. Reporter genes are commonly used to improve experimental accuracy. Typically, the “experimental” gene is correlated with the effect of specific experimental conditions, while the activity of the co-transfected “control” reporter gene provides an internal control, which serves as a baseline response. Normalizing the activity of the reporter gene minimizes experimental variability caused by differences in cell viability or transfection efficiency. For this work, the firefly luciferase pGL3-Control Vector (Promega) is used. Cells are harvested 48 hours after transfection, lysed and luciferase activity quantified using the Bright-Glo reagent (Promega) and Wallac Victor V plate reader. Amounts of the ots and AGT1 expression vectors are titrated to optimize transfection efficiency.

NIKS, NHDF and MSC cell populations transiently transfected with trehalose biosynthesis genes are incubated with media containing doxycycline (0, 1, 10, 100, 1000 ng/ml) to induce otsA and otsB expression; the cells are collected at 2, 6, 12, 24, and 48 hr after doxycycline addition for intracellular trehalose measurement. The percentage of the cells expressing the ots genes is limited by the transfection efficiency, therefore not all of the cells will be making trehalose. In addition, some cells will have taken up more DNA than others, so detection of trehalose will provide only a population average. Despite these caveats, detection of trehalose provides strong support for the function of the introduced genes.

NIKS, NHDF, and MSC cell populations are transiently transfected with the trehalose transporter genes with regulation by the Tet-On system such that synthesis of the transporter can be induced by addition of doxycycline into the culture medium. Twenty-four hours after transfection, NIKS cells and NHDFs are incubated with media containing doxycycline (0, 1, 10, 100, 1000 ng/ml) to induce expression of AGT1. At time points up to 24 hours after induction of transporter gene expression, the cultures are incubated in media containing 25 mM to 1 M trehalose for 10 to 60 minutes. Following this treatment, the cultures are washed with fresh medium, lysed, and quantified for intracellular trehalose.

The presence of intracellular trehalose is determined in using the enzymatic end-point assay for trehalose described by Kienle et al., Yeast 9(6):607-11(1993), in which trehalose is enzymatically hydrolyzed to glucose which is then detected via reduction of NAD as glucose-6-phosphate is oxidized to 6-phosphogluconate (Glucose (HK) Assay Kit, Sigma). The levels of endogenous glucose are subtracted out in a duplicate sample without enzymatic digestion of the trehalose. Positive and negative controls are prepared from cell culture medium with and without trehalose added at known concentration.

### Example 6

This example demonstrates that transiently transfected cells can survive air drying. The ability of engineered cells to survive in the dry state is evaluated in two ways for transiently transfected cell populations grown in submerged monolayer culture. The cultures are induced to synthesize trehalose or take it up from the medium as appropriate using levels of doxycycline chosen based on results of the preceding example. After a suitable trehalose accumulation period, the medium is removed from the monolayer cultures allowing them to air dry at room temperature. Cultures are dried for 30 minutes and held in the dry state for various periods up to 1 week.

It has been shown that function of dried platelets is greatly increased using an exposure to high relative humidity (RH) prior to rehydration. Thus, the rehydration procedure for these cultures is varied by exposing them to humidity levels ranging from 40% RH to 100% RH at temperatures from 25 to 37 C.

Following the hydration steps, cell survival is assessed using the ALAMARBLUE assay (Biosource International). ALAMARBLUE is a sensitive indicator of redox activity in actively metabolizing cells. It is useful in this work in that it is a non-destructive test of cell metabolic behavior post-drying. In this assay a standard curve is prepared by harvesting an actively growing plate of NIKS cells or NHDFs as appropriate and plating them at various densities bracketing the range of expected cell recovery from drying. These plates along with the dried/rehydrated test plates are then exposed to culture medium with 10% alamarBlue reagent added. The actively growing cells take up the alamarBlue dye and reduce it from its blue (oxidized) to red (reduced) form which is then free to pass back into the culture medium. Absorbance at 570 nm is measured for sampled medium every hour for the first 6 hours and then again at 24 hours to quantify recovery of cell metabolic activity.

As a longer-term indicator of complete functional recovery of dried cells, plates are cultured under standard conditions for at least 1 week post-drying. At the end of this period, colony formation is quantified as described elsewhere. Pegg et al., Cryobiology 44(1):46-53 (2002). Based on these results, the doxycycline exposure level and trehalose accumulation periods are re-evaluated to optimize the recovery of living cells from the dry state.

### Example 7

This example describes the development of trehalose-based high glass transition temperature solutions that have good biological compatibility, alleviate detrimental effects during freezing, and improve drying efficiency. The T_{g} of solutions with polymeric components (from 0.1 to 10%) including dextran, hydroxyethyl starch, polyvinylpyrrolidone, and polyvinylalcohol in combination with trehalose (from 20 to 60%) is measured. Sample preparation and T_{g} measurement by differential scanning calorimetry are described elsewhere. Miller et al., J. Phys. Chem. B. 103(46):10243 (1999).

Once the glass transition profiles of the trehalose:polymer mixtures have been determined, the most promising systems, i.e., those which combine high T_{g}, ease of preparation, no toxicity, and low cost, are combined with potassium phosphate salts to enhance vitrification. Potassium phosphate in particular is useful in that it undergoes relatively little pH drift with temperature, unlike sodium phosphate salts. Van den Berg et al., Arch. Biochem. Biophys. 81:319-329 (1959). The overall phosphate concentration will be varied with molar ratios of phosphate to trehalose from 0.1 to 2.

### Example 8

This example describes the development of procedures for freezing and drying modified cells. Development of a successful drying and freeze-drying process must consider a number of variables including duration of trehalose synthesis/uptake prior to preservation, optimal cooling rate, final frozen temperature, primary and secondary drying temperatures, and storage temperature/conditions (Table 1). The first cell type to be preserved will be MSCs. These cells represent an excellent test bed for the broad applicability of the technology developed in this proposal. They are immortal in culture and have both immediate commercial value as well as the potential to differentiate into multiple cell types (e.g., cartilage, bone, tendon). Thus, MSCs engineered to survive desiccation represent a commercially valuable product in themselves. In addition, these cells can readily be differentiated using commercial media into several daughter cell types. It is contemplated that a desiccation-resistant progenitor stem cell line can give rise to differentiated cells, tissues, and organs that are similarly desiccation-resistant. See Table 1 for a tabular summary of the test conditions.

TABLE 1
Experimental overview for freeze-drying development

| | Temperature | | Operating |
|---|---|---|---|
| Drying Phase | Range | Process Time | Pressure |
| Primary Drying | T_{g}−30° C. | To completion | 50 mtorr or |
| (sublimation | to T_{g} | | lower |
| of ice) | | | |
| Secondary Drying | Temperature | 10 hour hold at | Varied incre- |
| (diffusive remov- | increased | each temperature, | mentally from |
| al of water from | in 5° C. incre- | samples taken at | 50, 100, 250 |
| matrix) | ments up to | 1, 2, 5, 10 hours | mtorr using |
| | 25° C. | for analysis | dry N₂ gas |
| Air Drying | Varied from 4° | Samples taken at | Atmospheric |
| | C. to 37° C. | 1, 2, 6, 12, and | |
| | | every 12 hours | |
| | | thereafter for | |
| | | analysis | |

Freeze-Drying: Optimization of freeze-drying parameters is carried out on cells. The induction of trehalose synthesis/transport is tested at up to 60 minutes prior to removal from their tissue culture plate and freezing. Freezing rates are varied up to 20° C./min. The primary drying temperature is varied from the T_{g} of the solution to 30° C. below this value. Primary drying is carried out at low pressure until thermocouple readings indicate complete ice sublimation. In secondary drying, samples are held for 10 hours at temperatures beginning with the initial T_{g} and proceeding in 5° C. increments up to 25° C., to generate a T_{g} versus dryness curve. Samples from each experiment are evaluated for moisture, glass transition temperature, and viability. Miller et al., supra; Conrad et al., Cryobiology 41(1):17-24 (2000). This approach provides the thermodynamic data required to choose the most effective freeze-drying solutions, and the kinetic data needed to dry cells efficiently.

Air Drying: Given the recent results in air dried cells, we will evaluate this avenue of sample preservation. Cells will be cultured on a coverslip to 75% confluence. Induction of trehalose biosynthesis and AGT1 expression are carried out as above. Samples are immersed in preservation solutions for periods varying from 1 to 60 minutes and then air dried in a stream of dry nitrogen gas at various temperatures (Table 1). Samples are stored in a desiccator. Tissues are dried in their porous culture insert after induction and immersion as for the cell samples. Given the literature on imbibitional damage in dried systems, sample rehydration must be controlled. Crowe et al., Proc. Natl. Acad. Sci, USA 86(2):52-23 (1989). As a “standard” rehydration process, dried cells are rehydrated with growth medium and allowed to revive for 60 minutes at 37° C. Alternative rehydration protocols to be investigated include saline alone and 20% mannitol. Rehydration times are varied from 10 minutes to 24 hours at temperatures ranging from 0° C. to 37° C. Recent studies indicate that a humidification step is highly beneficial prior to rehydration of dried platelets. Wolkers et al., Cryobiology 42(2):79-87 (2001).

Assays: Cell and LSE viability are determined using the MTT assay for mitochondrial activity. Mosmann et al., J. Immunol. Meth. 65(1-2):55-63 (1983). Viability is quantified by comparison of OD₅₅₀ for preserved samples to that of appropriate controls. To establish long-term recovery, cells are plated according to standard procedures. After four days of growth, the plates are trypsinized and counted. To determine the survival of the various of cell layers in the LSE, confocal scanning laser microscopy is used. J. Biomed. Mat. Res. 30(3):331-9 (1996). Barrier function of LSEs is directly measured by surface electrical capacitance using a Dermaphase 9003 impedance meter (NOVA Technologies Corp). Boyce et al., J. Invest. Derm. 107(1):82-7 (1996). LSE histology is examined for cell differentiation (keratin-1, involucrin, filaggrin), proliferation (antigens to the antibody Ki-67), and apoptosis (active caspase-3) using methods detailed elsewhere. Loertsher et al., Toxicol. Appl. Pharmacol. 175(2):121-9 (2000). Cytokine response is compared pre- and post-preservation by culturing a biopsy of the fresh and preserved LSEs for 24 hours, sampling the growth medium, and analyzing it for the presence of VEGF, TGF-β, and IL-1α (all known cytokines involved in wound healing) by ELISA.

### Example 9

This example describes the constructions of vector for expression HVA1 and transfection of cells with the vector.

Expression Vector Constructs: As in the constructs for trehalose synthesis and uptake, the HVA1 gene is cloned into plasmids compatible with the Tet-On regulatory system. The HVA1 coding region is isolated by PCR using primers based on published sequences using a commercially-available barley cDNA (Stratagene). Straub et al., Plant Molec. Biol. 26(2):617-30 (1994). The HVA1 coding region is cloned into the tetracycline-responsive vector pTRE2. To enable selection of stable clones, a puromycin resistance expression cassette is cloned into the pTRE2 vector. Assays for transient expression of the HVA1 gene and efforts to generate stably transfected clonal cell lines are similar to those described in the preceding examples.

LEA Protein Synthesis in Monolayer Culture: Multiple clones that contain intact copies of the HVA1 gene are examined for protein expression in the presence of doxycycline. Expression is induced using a range of doxycycline levels, and cells are collected after 2, 6, 12, 24, and 48 hours for SDS-PAGE analysis and Western blotting. It is expected that different clonal lines express the genes at different levels and therefore will contain different steady-state levels of the HVA1 LEA protein. Thus, the profile of protein synthesis with varying doxycycline is repeated for 10 clonal cultures. Because the genes for trehalose synthesis/uptake and the HVA1 gene should be coordinately regulated by the Tet-ON system, cells are examinerd for trehalose accumulation and HVA1 protein synthesis at various time points.

Effect of LEA Protein on Air Dried Cells: A simple air-drying procedure is used to provide an early indication of the effectiveness of intracellular LEA proteins for drying of mammalian cells. Matsuo, J. Opthalmol. 85(5):610-12 (2001).

LEA Protein Biosynthesis in LSEs: LSEs are induced by doxycycline addition to the growth medium using the conditions that generate the highest level of LEA protein biosynthesis; tissues are incubated for 24-48 hours to allow for protein synthesis and accumulation. LSEs are harvested at 12, 24, 36, and 48 hours after doxycycline addition for SDS-PAGE analysis and Western blotting. A dose-response curve is generated to determine the minimum doxycycline dose required for maximal protein accumulation in each tissue.

### Example 10

This example describes the preservation of cells that express HVA1 and/or a trehalose synthesis pathway. This task will mirror closely the steps taken in Example 8 with the exception that the starting points for drying and freeze-drying will be the optimal conditions identified in Example 8. The difference between this Example and Example 8 is that that the samples of interest will have enhanced cytoplasmic vitrification tendency based on the expression of the HVA1 protein. For freezing steps, it is anticipated that faster freezing will become advantageous since the tendency for IIF will be reduced. For both freeze-drying and drying processes it is expected that the increased cytoplasmic vitrification will result in optimal product stability at higher moisture levels which means that drying cycles can be shortened. Furthermore, increased moisture will likely translate into more rapid recovery of cells and tissues from the dry state.

During freeze-drying optimization, primary drying temperature and pressure, secondary drying temperatures, times, and pressures are varied. Air drying parameters include dry gas flow rate and temperature. Finally, rehydration conditions for both drying approaches are performed as in Example 8.

### Example 11

This example describes the optimization of parameters for preservation of skin equivalents. Apoptosis has been described in the literature as a potentially significant source of cell loss following cryopreservation. Mathew et al., In Vitro & Mol. Toxic. 12(3):163-172 (1999). As has been reported, one approach to reducing apoptosis is to add inhibitors to early or late stage apoptotic enzymes such as caspase-9 or caspase-3, respectively. These inhibitors are commercially available (Calbiochem) and have shown benefit in recovery from cryopreservation and drying. Baust et al., In Vitro Cell. & Devel. Biol. Anim. 36(4):262-70 (2000). Accordingly, caspase inhibitors are added to the preservation medium at various time points prior to freezing or drying. The effect of the capsase inhibitors is assessed using histological markers for fresh and preserved LSEs as described above.

The approach of using caspase inhibitors has been demonstrated in isolated cells, but delivery of these compounds to cells in a tissue is challenging. Thus, the use of siRNA in inhibiting the apoptotic pathway has been evaluated. In this experiment NIKS cells were transiently transfected with two forms of luciferase, firefly and rinella. An siRNA molecule against the firefly luciferase was then introduced to the cells using the TransIT-TKO reagent from Mirus, and it was found that 90% reduction of the target gene expression was achieved. It is reasonable to expect this effect to last for ten days to two weeks. Thus, as a secondary approach to apoptosis reduction, cells in monolayer culture are transfected with siRNA directed at the caspase-3 gene just prior to seeding the cells onto the LSE. In 14 days the LSE will be mature and ready for preservation with the siRNA still functional in the cells. After rehydration the tissue should be greatly limited in its ability to apoptose. Given that this effect will be transient, it is much safer than genetically knocking out apoptotic genes that has been strongly correlated to tumor formation.

Gene Array Analysis: Gene array analysis is used to monitor global changes in gene expression following recovery from the dried state, RNA from rehydrated and control (not preserved) tissue is submitted to Genome Explorations, Inc., which will perform gene expression array hybridization and data analysis. Biotinylated cDNA probes is generated from the RNA samples and is hybridized to the U133 GeneChips from Affymetrix, which represent transcripts from approximately 33,000 independent genes. After normalization of each sample to a set of control RNAs, genes whose expression is increased or decreased by the preservation techniques can be determined. Of particular interest are genes involved in scarring (extracellular matrix molecules, extracellular proteases), vascularization, wound healing, and apoptosis.

In vitro Evaluation of Successful Preservation: LSEs are rehydrated according to the methods in the preceding example and cultured for two weeks. During this time period, any delayed cell damage becomes evident and helps to distinguish between the preservation methods we have developed to this point. On every second day after rehydration, replicate cultures are sacrificed for analysis. Overall viability is determined using the MTT assay for mitochondrial activity (supra). To determine the survival of the different cell layers in the LSE, confocal scanning laser microscopy is used. Barrier function of LSEs is directly measured by surface electrical capacitance (supra), and histology is examined for cell differentiation, proliferation, and apoptosis as above. Cytokine response is compared pre- and post-preservation by culturing a biopsy of the fresh and preserved LSEs for 24 hours, sampling the growth medium, and analyzing it for the presence of VEGF, TGF-β, and IL-1α (all known cytokines involved in wound healing) by ELISA.

In vivo Evaluation of Successful Preservation. Preserved LSEs using modified NIKS keratinocytes and NHDFs are suitable for grafting onto nude mice. Grafting protocols based on methods developed at the University of Wisconsin by Dr. Lynn Allen-Hoffmann (Dept. of Pathology) and Michael Schurr, M.D. (Associate Professor, Dept. of Surgery) will be used to assess in vivo survival and function of preserved LSEs. Barrier function of grafted LSEs will be measured daily by surface electrical capacitance. Assessment of grafting success at 7 and 14 days post-surgery is completed by the immunohistological analysis of biopsies taken from the graft site. As for the in vitro studies, histological sections are evaluated for proper cell differentiation, proliferation, and apoptosis. Grafted animals are monitored for any long-term indications of retention, rejection, replacement, antigenicity or unexpected growth behavior of the graft for six months. After the study, test animals are sacrificed and examined for any sub-chronic toxicity that might not be otherwise evident.

### Example 12

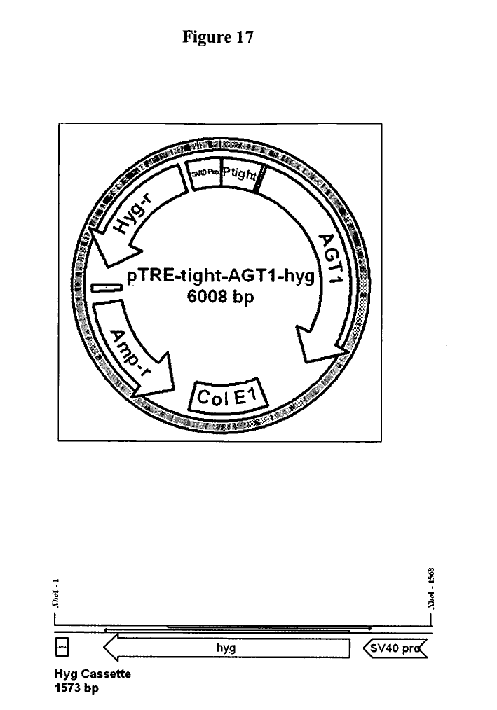

This example describes the construction of the pTRE-tight-AGT1-hyg vector, which expresses the AGT1 gene (FIG. 17). The construction of this vector used an XhoI cleavage site proximal to the 5′ end of the Ptight promoter. A second XhoI cleavage site was present near the 5′ end of the bacterial origin of replication sequence in the base vector (Col E1). Thus, a partial digest of the base vector was first performed, and product plasmid cleaved at the second XhoI was isolated. The ends were blunted, and the vector was re-ligated. This process eliminated the second site allowing for the insertion of the hygromycin cassette (FIG. 17) using an XhoI digest and ligation.

Hygromycin resistance can be conferred by expression of the resistance gene under control of the constitutive SV40 promoter. An SV40 polyadenylation sequence is added to facilitate transgene expression in mammalian cells.

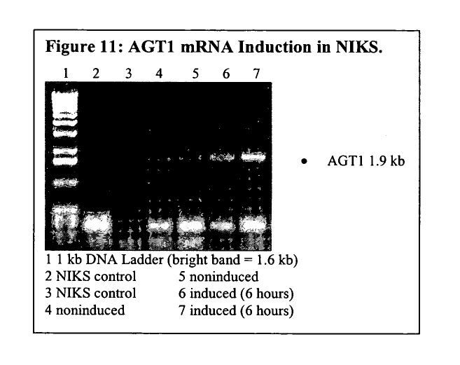

Expression of the AGT1 gene in NIKS cells was demonstrated through detection of its mRNA. An agarose gel demonstrating the RT-PCR result for AGT1 mRNA expression is shown in FIG. 11. In the gel a single band is present at 1.9 kb which is the expected size of AGT1 mRNA.

### Example 13

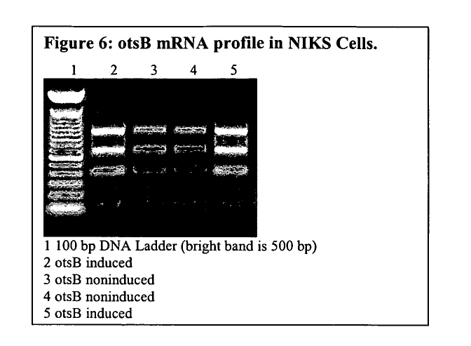

This example describes the finding of two cryptic splicing sites in the otsB gene product and the presence of several introns in the otsA gene. The presence of these splicing events is mentioned nowhere in the literature due to the fact that reports to date dealing with otsA and B in mammalian cells have not examined expression at the RNA level. In the case of otsB, the cryptic splicing occurs between sites in the otsB gene itself and the rabbit β-globin poly-adenylation (polyA) sequence downstream of it. The use of a poly-adenylation sequence downstream of a transgene is known to terminate translation and increase the stability of the transgene mRNA. Accordingly, a downstream poly-adenylation sequence was added to each transgene in the pBI-otsAB-hyg construct; the otsB gene is followed by the rabbit β-globin polyA sequence, which is known to contain an intron. This polyA intron is useful in that it improves the stability of the mRNA, aids in its efficient translation, and simplifies mRNA detection by Reverse Transcriptase-PCR (RT-PCR). In this case, the intron acceptor site, however, apparently caused cryptic splicing at two sites within the otsB gene as well as demonstrated by RT-PCR results for otsB in NIKS cells (FIG. 6).

FIG. 6 presents a RT-PCR result for otsB gene expression in NIKS cells. The cells were transfected with the pBI-otsAB-hyg construct using Trans-IT Keratinocyte reagent from Mirus (Madison, Wis.). Expression was induced for 24 hrs with doxycyline, and the mRNA was harvested. The sample was then treated with DNAse to remove genomic DNA and reverse transcriptase to derive cDNA from the mRNA. This cDNA was then PCR amplified using an otsB specific forward primer and a β-globin polyA reverse primer.

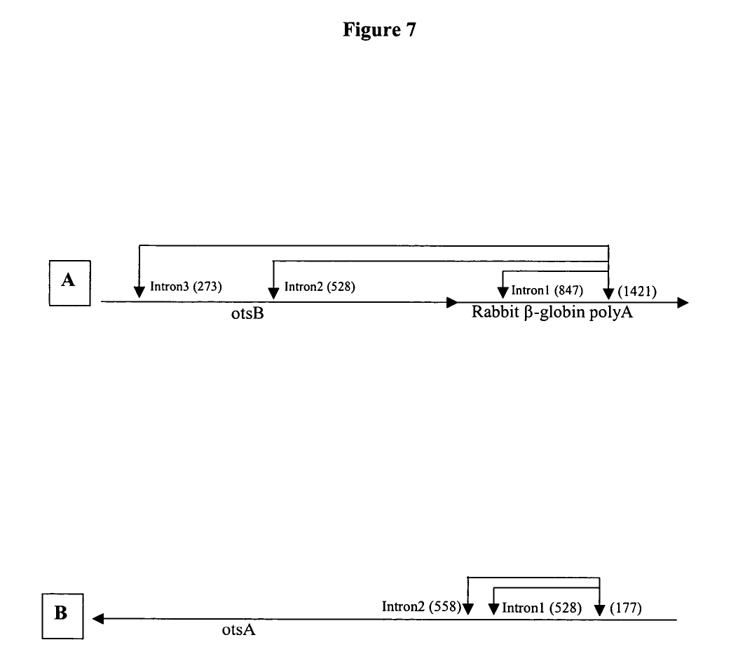

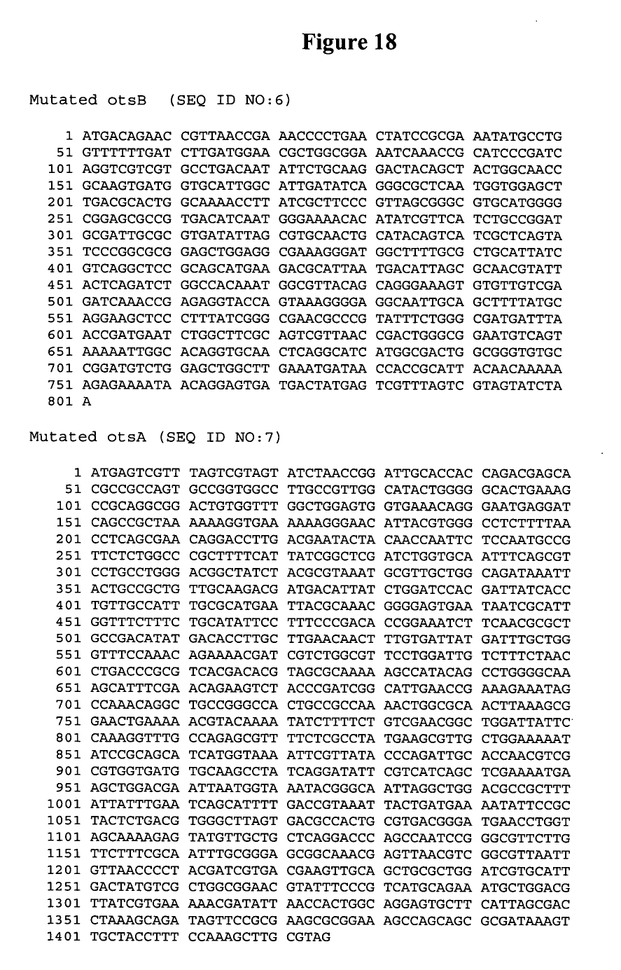

In FIG. 6, three bands are evident. Each band represents a different mRNA splicing product. To confirm the location of the splicing events, each band was excised from the gel, purified, ligated into a cloning vector, transformed into E. coli, grown up, purified once again, and sequenced. The sequencing results indicate that the splicing pattern is as shown in FIG. 7A. The Intron1 product in FIG. 6 is the expected mRNA for otsB with the processed β-globin polyA. In addition, mRNA representing cleavage at two intron donor sites within the otsB gene itself appeared (Intron2 and Intron3 products). The presence of these alternative splicing products is not likely to interfere with the function of the correct otsB mRNA but they will lower the protein levels achieved. The splicing was eliminated by site-directed mutagenesis. This was accomplished by making degenerate single base pair changes from GGT (Gly) to GGG (Gly) within the sequence at the intron donor sites for the Intron3 and Intron2 sites. This change was made and verified by sequencing. The mutated otsB sequence (SEQ ID NO:6) is shown in FIG. 18.

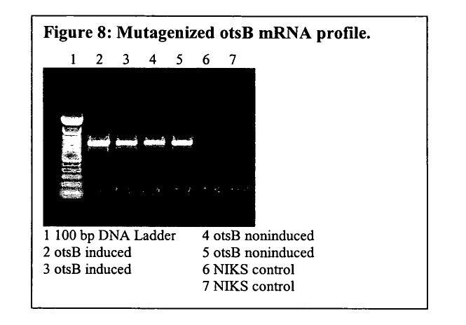

After identifying the cryptic splice sites and eliminating them via site-directed mutagenesis of the otsB gene in the pBI-otsAB-hyg construct (designated pBI-otsAB*-hyg), a confirmatory transfection was performed in NIKS cells with followup RT-PCR analysis (FIG. 8). (Note: Similar analysis was performed in NHDFs and MSCs with similar results.) Given the single mRNA band of the anticipated size for otsB with the spliced polyA sequence, it was concluded that the unexpected splicing of the otsB mRNA was eliminated.

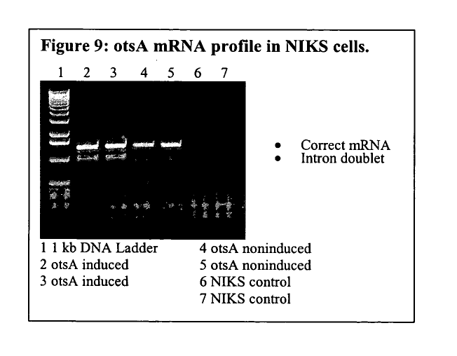

Concurrent with the adjustments to the otsB gene, it was found that the otsA gene also contains unexpected and undesirable intron splicing (FIG. 9). In this case, intron splicing was found to occur entirely within the otsA gene itself and was not a result of the flanking regions. This phenomenon has not been reported in the literature. In order to eliminate the aberrant splicing, the cDNA bands were excised from the agarose gel, purified, ligated into a cloning vector, transformed it into E. coli, and isolated for sequencing. The results are shown in FIG. 7B. Two introns were identified with a common donor site at 177 bp from the start of the otsA gene. The acceptor sites were closely grouped at 528 and 558 bp and appear as a poorly defined doublet in the gel in FIG. 9.

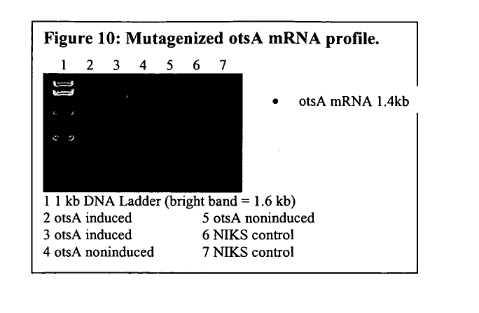

Once again site-directed mutagenesis was employed to eliminate the detected splicing events. In this case, a GGT (Gly) was converted to a GGG (Gly) for the donor site, and a CAG (Gln) was converted to a CAA (Gln) for both the acceptor sites. The base pair changes were confirmed by sequencing analysis. The mutated otsA sequence (SEQ ID NO:7) is shown in FIG. 18. RT-PCR testing of cells transfected with the pBI-otsAB-hyg construct mutagenized in both otsB and otsA genes (designated as pBI-otsA*B*-hyg) gave rise to the anticipated bands for otsB as well as for otsA (FIG. 10). However, in FIG. 10 a very light secondary band persists in the agarose gel at a size of roughly 1 kb. This band is likely due to the presence of an additional alternative splicing product which may have been present in the original samples but was not readily apparent due to the prevalence of the other splicing products.

### Example 14

This example describes the demonstration of gene expression for the otsA and otsB genes in both NHDFs and MSCs. Cell samples were transfected with the appropriate TetON control plasmid and either the pBI-otsAB or pTRE-tight-AGT1 response plasmids. Gene expression was detected using RT-PCR as follows. The transfected cells were induced with doxycycline, and the resulting RNA was harvested. Interfering DNA was eliminated by DNase digestion as a first step. The remaining RNA was then converted back to cDNA using reverse transcriptase. The resulting cDNA was amplified by PCR using primers specific to the genes of interest.

### Example 15

This example describes trehalose synthesis in NIKS cells. Experiments were carried out by transfecting NIKS cells with the K14-TetON control plasmid and pBI-otsAB response plasmid in a stoichiometric ratio of 1:9 with Trans-IT keratinocyte reagent as described above. At 24 hours post-transfection the expression of the exogenous genes was induced for various amounts of time with 200 ng/ml doxycycline to allow for synthesis and accumulation of intracellular trehalose. To quantify intracellular trehalose, cells were harvested from their culture dish, simultaneously counted and sized using the Beckman-Coulter Multisizer3, centrifuged into a pellet, and lysed by triple freeze-thaw cycles. The lysate was then analyzed by HPLC using the method described below. A summary of several of these experiments is presented in the following table.

TABLE 2
Summary of Inducible Trehalose Synthesis
Experiments in NIKS Cells.

| | | Intracellular |
|---|---|---|
| | | Trehalose |
| Expt. | Description | Synthesized |
| 1 | Cells transfected for 1 day; | 0.8 mM |
| | induced for 1 day | |
| 2 | Repeat of 1 | 0.4 mM |
| 3 | Repeat of 1 with increased glucose | 1.3 mM |
| | and insulin in the medium | |
| 4 | Cells transfected for 1 day; | 0.4 mM |
| | induced for 2 to 4 days | |
| 5 | Repeat of 1 using DMSO to extract | 0.3 mM |
| | trehalose instead of freeze-thaw | |
| | Same as above except control:response | 0.6 mM |
| | ration was 1:1 | |

Throughout the various experiments, transfected NIKS cells accumulated trehalose to an intracellular concentration in the range from 0.3-1.3 mM. Based on an averaged cell volume of 3200 μm³ commonly observed for NIKS cells, this represents 0.33-1.42 pg of trehalose per cell. This level compares well with most other published results as shown in Table 3 below.

TABLE 3
Summary of Reported Intracellular Trehalose Accumulation.

| | | Trehalose |
|---|---|---|
| Report | Cell Type | (pg/cell) |
| Levine et al, Nature Biotech 2000 | 293 Cells | 0.5 |
| Chen et al, J. Biol. Chem. 2003 | 293 Cells | 60 |
| Levine et al, Cryobiology 2001 | NHDFs | 1.7 |
| de Castro and Tunnacliffe, FEBS | LMTK⁻ | 40 |
| Letters 2000 | | |

Experiments were conducted to identify possible factors that might be limiting trehalose synthesis. One possibility was the expression or induction of trehalase enzymatic activity in response to trehalose synthesis and accumulation. Trehalase is known to be present in mammalian systems with expression localized to the intestinal epithelia and the kidneys. Thus, it was possible, however unlikely, that the transfected cells could be responding to the presence of trehalose by activating this degradative pathway.

To investigate this area RNA was harvested from transfected cells for analysis by RT-PCR. Trehalase mRNA was not detected under any condition tested. While this result does not rule out the possibility of trehalose breakdown via endogenous, broad-specificity hydrolase activity in the cells, it does confirm that the cells are not responding to the presence of the trehalose itself in an adverse way.

### Example 16

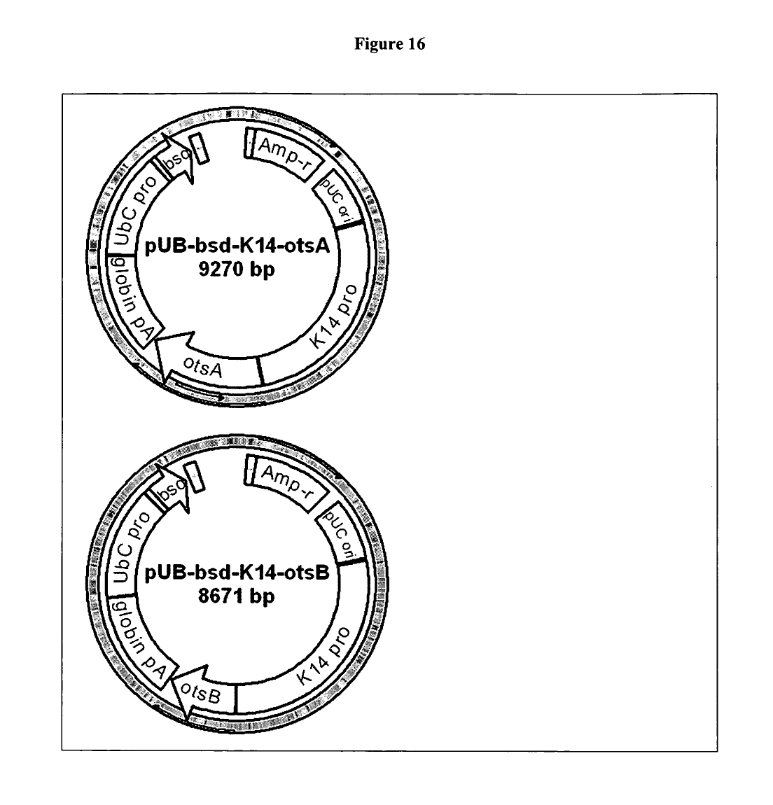

This example describes a comparison of trehalose accumulation through inducible (TetON) and constitutive systems. Two vector constructs were assembled using the keratin-14 (K14) promoter to regulate expression of otsA and B. K14 is a highly expressed protein in keratinocytes growing both in monolayer and organotypic culture. As shown in FIG. 16, two vectors were assembled from the same base construct. A K14 promoter is used to drive expression of the otsA or otsB genes. The genes are followed by a rabbit β-globin polyadenylation sequence as in the response vector constructs. The constructs also contain the ubiquitin-C promoter, which drives the expression of a blasticidin resistance gene.

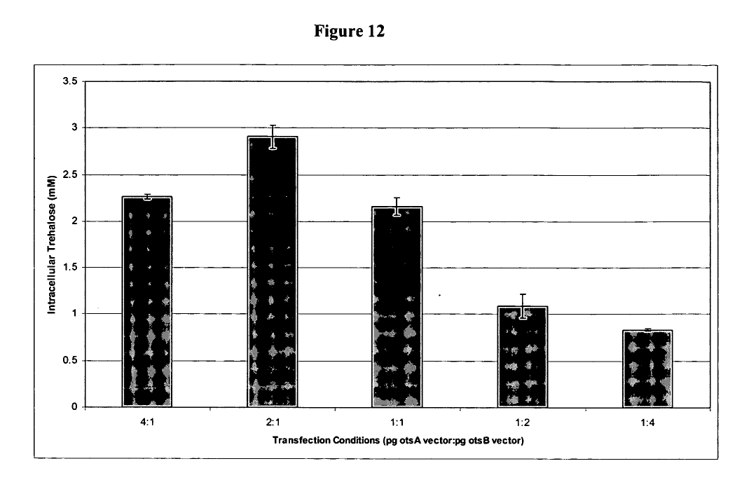

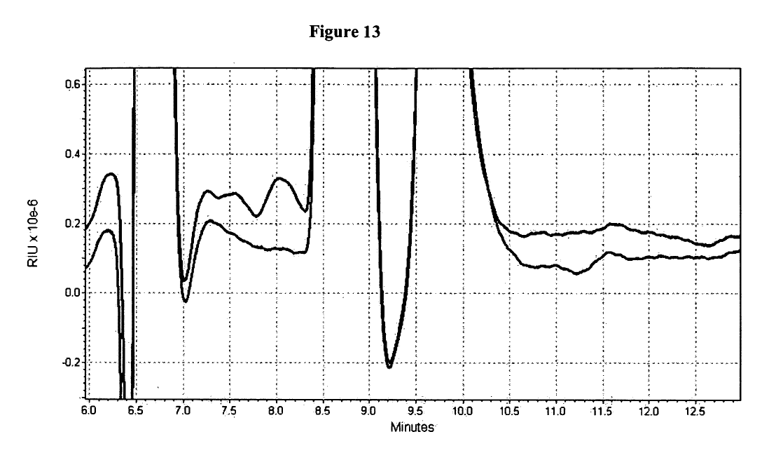

Differing levels of the otsA construct relative to otsB were compared. It is not possible to adjust this variable with the TetON system. As in other transfections, the NIKS cells were grown from frozen stocks, passed to tissue culture dishes at 10⁶ cells per 55 cm² plate (without feeder cells present), transfected with the vectors using TransIT-Keratinocyte reagent and harvested 24 hours later. Cells were centrifuged into a pellet and lysed by triple freeze-thaw cycles. Cell lysate was filtered and analyzed by HPLC. The results are shown in FIG. 12. As the results indicate, the optimal transfection condition in this experiment included a mass ratio of 2:1 between the otsA bearing construct and the otsB construct. The present invention is not limited to a particular mechanism. Indeed, an understanding of the mechanism is not necessary to practice the present invention. Nonetheless, it is contemplated that the difference is due to inherent differences in the efficiency of expression of these two genes and the relative activities of the two enzymes. This result has been repeated several times. Furthermore, the trehalose accumulation period has been extended to 48 hours using the same 2:1 ratio of vectors. These results are summarized in Table 4 below. In addition, the identity of the quantified HPLC peak as trehalose was confirmed through the use of trehalase digestion as shown in FIG. 13.

TABLE 4
Maximal Trehalose Accumulation under Constitutive Expression.

| Experiment | 24 hours | 48 Hours |
|---|---|---|
| 1 | 2.8 mM | — |
| (above) | | |
| 2 | 2.5 mM | — |
| 3 | 2.1 mM | 6.5 mM |
| 4 | 3.6 mM | 4.7 mM |
| 5 | — | 6.4 mM |

Based on these results it is clearly possible for the NIKS cells to reach 5 mM intracellular trehalose with constitutive expression of the otsA and B genes.

### Example 17

This Example describes experiments conducted on NIKS cells transfected with the gene for the AGT1 trehalose transport protein under the control of the TetON inducible system. It is contemplated that the extracellular environment plays a role in activating the transporter. This transporter is native to yeast where it was first identified as a maltose transport protein. It has been characterized with a maximum in transport activity at pH 5 with a Kₘ of 4 mM trehalose. This example describes an investigation of conditions that give rise to maximal trehalose uptake in mammalian cells.

Effect of pH changes: As a starting point for introducing trehalose to cells via the AGT1 transporter, the extracellular pH was lowered. Several experiments were conducted in which the NIKS cells, NHDFs, and MSCs were each exposed to saline buffered with sodium citrate at pH 5 for various lengths of time to verify that the pH exposure itself would not cause significant cell damage. After exposure the cells were returned to normal medium and allowed to continue to grow for one day. After that time, cells were treated with MTT reagent, a well characterized indicator of metabolic activity, to quantitate any changes in culture viability due to the pH shock. The results are outlined in the table below. The exposure of all cell types to pH 5 for periods less than about 30 minutes reduced the culture viability by no more than 10%. In the case of MSCs, no reduction in viability was detected even after 1 hour of pH 5 exposure.

Effect of brief pH Shock on Cell Viability after 24 hours

| | Minutes of pH 5 exposure | | | | |
|---|---|---|---|---|---|
| Cell | 0 | 5 | 15 | 30 | 60 |
| NIKS | 100% | 86% | 93% | 90% | 81% |
| NHDF | 100% | 102% | 94% | 90% | 86% |
| MSC | 100% | 111% | 113% | 100% | 108% |

Effect of pH on Trehalose Uptake: As in the case of trehalose synthesis, the initial studies in trehalose uptake were carried out in NIKS cells. The experiments were carried out as for trehalose synthesis with the exception that an uptake step is included. That is, the cells are exposed for a period of time to extracellular trehalose, which is later washed away prior to cell harvest and lysis.

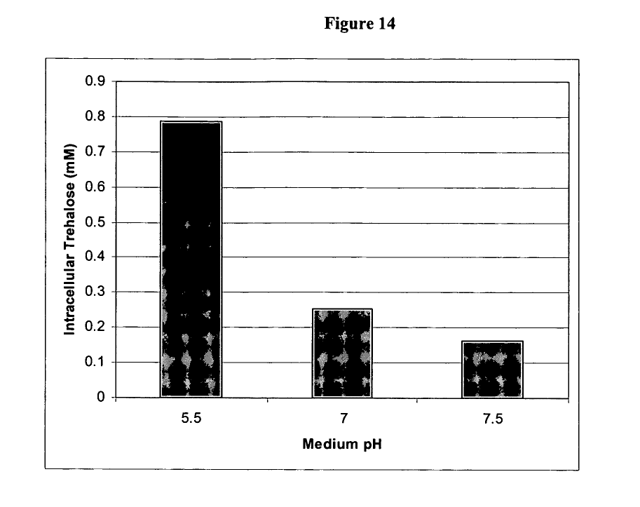

The concentration of extracellular trehalose used was chosen from an earlier experiment in which 48 hours of exposure to 50 mM extracellular trehalose was found to have no impact the growth of NIKS cells whereas 100 mM retarded cell growth by 50. Functional assessment of the AGT1 transporter was examined as follows. NIKS cells were transfected as for trehalose synthesis (See above). NIKS cells were transfected with K14-TetON (control) and pTRE-tight-AGT1 (reponse) plasmids per standard protocol. The samples were selected for 1 day with blasticidin to enrich for transfected cells and passed to new plates with feeder cells. After 24 hours the cells were induced with doxycycline for an additional 24-48 hours. The cells were then exposed to uptake medium consisting either of culture medium or a buffered saline with 50 mM added trehalose. After uptake the cells were washed, harvested, lysed and their intracellular trehalose determined by HPLC. The results from one such experiment are presented in FIG. 14. In this case the pH of the uptake medium was varied. The apparent trehalose uptake is higher at reduced pH where the AGT1 protein (a proton symporter) is more active.

In some embodiments, in order to effectively characterize trehalose delivery via the AGT1 transporter, a K14-AGT1 gene vector construct is used. In the uptake experiments, despite diligent washing some extracellular trehalose is carried forward through the lysis steps. The result is that even non-transfected cells if exposed to trehalose appear to contain some intracellular sugar. The present invention is not limited to a particular mechanism. Indeed, an understanding of the mechanism is not necessary to practice the present invention. Nonetheless, it is possible that trehalose may be entering the cells at some low level by an alternate endogenous pathway. In yeast, a second disaccharide transporter with much lower specificity (Kₘ=100 mM) is known to exist. Thus, in some embodiments, the experimental protocol is modified to further reduce the chance of trehalose being carried forward into the cell harvest and lysis steps thereby improving experimental sensitivity.

### Example 18

This Example describes dye uptake studies using pNPαG, a marker molecule that is essentially a nitrophenol ring attached to a disaccharide unit. The transporter (such as AGT1) delivers the pNPαG to the cytoplasm where non-specific hydrolases split the two moieties. The nitrophenol group after cleavage becomes a strong chromophore at a wavelength of 400 nm. Thus, the appearance of absorption at 400 nm can be taken as a direct indication of sugar uptake.

It was first determined if NIKS cells have the required hydrolase activity to split the pNPαG after its transport into the cells. The effect of cell lysate on pNPαG in solution was evaluated. If the needed hydrolases exist, then cleavage of pNPαG would be detected when exposed to lysed cells. NIKS cells were grown in tissue culture to confluence and lysed with GloLysis Buffer (Promega). This buffer, typically used for luciferase assays, is optimized for cell lysis without harming sensitive enzymes. pNPαG at a final concentration of 5 mM was added to samples of the cell lysates and also to a sample of the lysis buffer without cells. The optical density at 400 nm (OD₄₀₀) was measured over time.

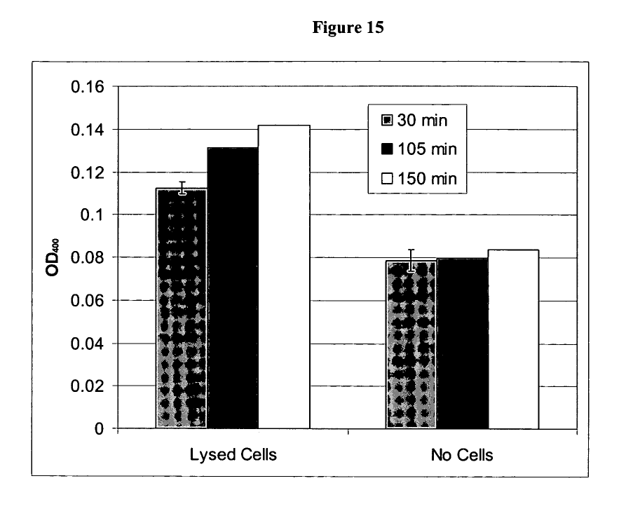

From the results in FIG. 15, it appears that lysed NIKS cells contain an adequate level of hydrolase activity to cleave the dye at detectable levels in a reasonable amount of time.

### Example 19

This Example describes the development of an assay for characterization of trehalose synthesis and/or uptake in cells. Two carbohydrate columns were evaluated for the detection of trehalose using a Beckman System Gold HPLC with Refractive Index (RI) detection: the Phenomenex Luna NH2 column and the Bio-Rad Aminex-87H column. An advantage of the Phenomenex column is its ability to run in both normal (for carbohydrate detection) and reversed phase mode (for proteins). Another advantage of this column is its faster flowrate allowing for higher sample throughput. However, after extensive system testing and troubleshooting, it was concluded that the column packing (or the acetonitrile-water mobile phase) was subject to minute pressure fluctuations which resulted in unacceptably noisy baseline RI signals.

The Aminex column uses an aqueous mobile phase and delivered much more stable baselines. Initial work followed the manufacturer's recommendation of 5 mM H₂SO₄ in water as the mobile phase. This was increased this level to 25 mM H₂SO₄ to avoid retention time drift due to inorganic salts in the samples (see trehalase section below). Trehalase Digestion—A low level of interference from an unknown species that nearly coelutes with trehalose is present. Several approaches were used to eliminate this interference. First, alterations to the mobile phase were analyzed. The use of an enzymatic trehalose digest was also tested. The identity of a trehalose peak has been confirmed in the literature by using this approach. Trehalase (from porcine kidney) was used to identify and quantify trehalose levels. The Na-citrate buffering system used in the trehalase digest protocol caused interference and replacement of the immobilized protons on the Aminex stationary phase which resulted in significant peak migration from injection to injection. This effect was countered by increasing the acidity of the mobile phase.

Mobile Phase Considerations—As mentioned above, initial tests indicated the presence of the co-eluting peak, which necessitated evaluating trehalose quantification by enzymatic digest. This digest procedure as reported by Levine is carried out in a sodium citrate buffer at pH 5.7. The presence of the sodium in the buffering system caused interference with the stationary phase of the Aminex column. That is, the sodium ions were apparently replacing hydrogen, which is ionically bound to the column packing with the result that the trehalose retention times increased significantly in a monotonic fashion. To counter this effect, the acid strength of the mobile phase was adjusted to 25 mM H₂SO₄. This change eliminated the peak drift, and, furthermore, it helped to separate the trehalose peak from the unknown co-eluting species.

The final conditions for the trehalose detection assay are as follows:
Column—Bio-Rad Aminex 87H (4.6×250)
Mobile Phase—25 mM H₂SO₄ in water
Flow—0.6 mL/min
Temperature—65 C
Detection—Refractive Index.
   With the increased resolution of the trehalose peak in the higher acid mobile phase, it is not necessary to use the enzymatic digest approach for trehalose quantitation.

Cell Lysis Procedures—To enhance the ability to detect trehalose in engineered cells, a variety of the reported methods for cell lysis were evaluated. These have included extraction of trehalose using chemical agents such as perchloric acid, trichloroacetic acid, ethanol, and sodium hydroxide, and physical cell lysis methods such as boiling and freeze-thaw. It was determined that a simple 3× freeze-thaw cycle was effective at allowing for complete release of trehalose. No degradation of spiked trehalose in these lysate samples was observed over time.

### Example 20

This example describes an investigation of to what extent a mixed population of NIKS containing K14-otsA-globin and K14-otsB-globin vectors, respectively, can synthesize trehalose. Results were compared to trehalose synthesis in NIKS containing both K14-otsA-globin and K14-otsB-globin in a ratio of 2:1. Additional controls were NIKS with K14-otsA-globin or K14-otsB-globin.

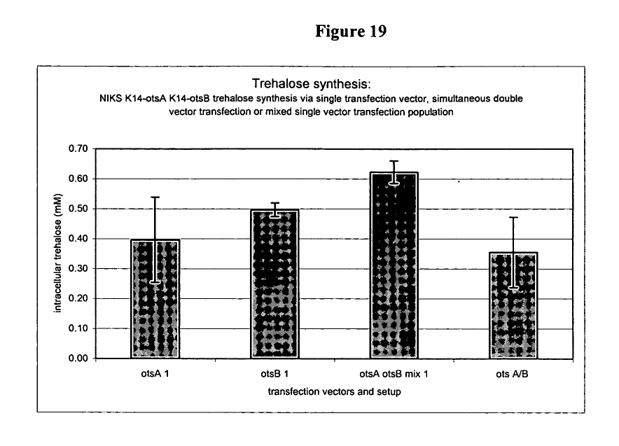

As in other work with NIKS cells (see above), the cells were thawed from frozen stock and plated onto 3T3 feeder cells in 55 cm² dishes. Cells were cultured for 5 days. At this point, the cells were harvested from their plates and the K14 otsA-globin and k14-otsB-globin vectors were introduced into the cells either separately or in combination as above. The cells were then plated in duplicate onto feeder cells in 55 cm² dishes as follows:
1. Untransfected control cells
2. NIKS transfected with K14-otsA-globin
3. NIKS transfected with K14-otsB-globin
4. NIKS transfected with both K14-otsA-globin and K14-otsB-globin
5. A mixture of 2) and 3) above
   After two days of continued cell culture, the NIKS cells were harvested and analyzed for intracellular trehalose content. Total cell numbers and average cell volumes were determined using a Beckman-Coulter Multisizer 3 Counter. Cells were spun down and the pellets were frozen at −20° C. Pellets were thawed and 150 μl DMSO was added to extract trehalose. Samples were transferred to a 1.8 ml centrifuge tube and spun down for 10 minutes at 12,000×g. The supernatant was analyzed using a Beckman-Coulter System Gold HPLC with a Bio-rad HPLC Organic Acid Analysis Column (AMINEX HPX-87H Ion Exclusion) column at 0.6 ml/min, 65° C. column temperature, a 25 mM H₂SO₄ in HPLC grade water mobile phase, with refractive index detection to determine trehalose concentration. Intracellular trehalose was then calculated based on HPLC peak areas from a trehalose standard curve run in DMSO, the DMSO extract volume, the total cell number, and the individual cell volume as shown below. The results are shown in FIG. 19.
   trehalose concentration [mmol/ml] in DMSO=(peak area×1.17693E-06−0.000319101) [mg/ml]/342.3 [mg/mmol]
   Intracellular trehalose concentration [mM]=(trehalose conc.)×(volume of DMSO extract)/(cell number×volume per cell).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, biochemistry, or related fields are intended to be within the scope of the following claims.

1. A mammalian cell comprising a gene encoding a trehalose transport protein.

2. The mammalian cell of claim 1, wherein said trehalose transport protein is AGT1.

3. The mammalian cell of claim 1, wherein said trehalose transport protein is operably linked to a promoter.

4. The mammalian cell of claim 3, wherein said promoter is selected from the group consisting of an inducible promoter and a constitutive promoter.

5. The mammalian cell of claim 1, wherein said cell is stably transfected.

6. The mammalian cell of claim 1, wherein said cell is a keratinocyte.

7. The keratinocyte of claim 6, wherein said keratinocyte is an immortalized keratinocyte.

8. The keratinocyte of claim 6, wherein said keratinocyte is a NIKS cell.

9. The mammalian cell of claim 1, wherein said cell is selected from the group consisting of stem cells, tissue culture cells, primary culture cells, and immortalized cells.

10. An organ comprising the mammalian cell of claim 1.

11. The organ of claim 10, wherein said organ is skin.

12. The organ of claim 10, wherein said organ is selected from the group consisting of skin, heart, liver, pancreas, kidney and lung.

13. The organ of claim 10, wherein said organ is dried.

14. The organ of claim 13, wherein said organ is freeze dried.

15. The organ of claim 13, wherein said organ is air dried or vacuum dried.

16. The cell of claim 1, wherein said cell is dried.

17. The cell of claim 16, wherein said cell is freeze dried.

18. The cell of claim 16, wherein said cell is air dried or vacuum dried.

19. A kit comprising the mammalian cell of claim 1 and instructions for its use.

20. A kit comprising the organ of claim 10 and instructions for its use in treating a patient.

21. A mammalian expression vector comprising a gene encoding a trehalose transport protein operably linked to a promoter functional in mammalian cells.

22. A mammalian cell comprising on or more genes encoding a trehalose synthesis pathway.

23. The mammalian cell of claim 22, wherein said genes encoding a trehalose synthesis pathway comprise otsA and otsB.

24. The mammalian cell of claim 23, wherein said otsA gene has the nucleic acid sequence of SEQ ID NO:7 and said otsB gene has the nucleic acid sequence of SEQ ID NO:6.

25. The mammalian cell of claim 23, wherein said otsA and otsB genes are in at least one expression vector, and wherein said otsA and otsB genes are operably linked to at least one promoter.

26. The mammalian cell of claim 25, wherein said promoter is selected from the group consisting of an inducible promoter and a constitutive promoter.

27. The mammalian cell of claim 25, wherein said otsA and otsB genes are on two separate expression vectors.

28. The mammalian cell of claim 27, wherein said two separate expression vectors are present at a ratio of approximately two otsA containing expression vectors to 1 otsB containing expression vector.

29. The mammalian cell of claim 23, wherein otsA and otsB gene functions are on a single gene.

30. An organ comprising the mammalian cell of claim 22.

31. A human skin equivalent comprising the mammalian cell of claim 22.

32. The human skin equivalent of claim 31, wherein said human skin equivalent is dried.

33. The human skin equivalent of claim 31, wherein said human skin equivalent is freeze dried.

34. The human skin equivalent of claim 31, wherein said human skin equivalent is air or vacuum dried.

35. The mammalian cell of claim 22, wherein said cell is dried.

36. The mammalian cell of claim 22, wherein said cell is freeze dried.

37. The mammalian cell of claim 22, wherein said cell is air dried or vacuum dried.

38. A kit comprising the human skin equivalent of claim 22 and instructions for its use in treating a patient.

39. A method of preserving mammalian cells comprising
a) providing cells comprising a gene encoding a trehalose transport protein;
b) culturing said cells under conditions such that said gene encoding a trehalose transport protein is expressed and trehalose is taken into said cells; and
c) drying said mammalian cells.

40. The method of claim 39, further comprising the step of freezing said cells prior to drying.

41. The method of claim 39, wherein said cell is stably transfected.

42. The method of claim 39, wherein said cell is a keratinocyte.

43. The method of claim 39, wherein said cell is a NIKS cell.

44. The method of claim 43, wherein said NIKS cell is stratified.

45. The method of claim 39, wherein said mammalian cells are in an organ.

46. The method of claim 45, wherein said organ is skin.

47. The method of claim 45, wherein said organ is a human skin equivalent.

48. The method of claim 45, wherein said organ comprises NIKS cells.

49. The method of claim 45, wherein said organ comprises stratified NIKS cells.

50. The method of claim 39, wherein said drying comprises freeze drying.

51. The method of claim 39, wherein said drying comprises air or vacuum drying.

52. The method of claim 39, wherein said culturing said cells is performed at a pH of about 5.5 or lower.

53. A method of preserving mammalian cells, comprising:
a) providing mammalian cells comprising one or more genes encoding a trehalose synthesis pathway;
b) culturing said mammalian cells under conditions such that said genes encoding a trehalose synthesis pathway are expressed; and
c) drying said mammalian cells.

54. The method of claim 53, further comprising the step of freezing said cells prior to drying said cells.

55. The method of claim 53, wherein said trehalose synthesis pathway comprises the otsA and otsB genes.

56. The method of claim 55, wherein said otsA gene has the nucleic acid sequence of SEQ ID NO:7 and said otsB gene has the nucleic acid sequence of SEQ ID NO:6.

57. The method of claim 55, wherein said otsA and otsB genes are in at least one expression vector, and wherein said otsA and otsB genes are operably linked to at least one promoter.

58. The method of claim 57, wherein said promoter is an inducible promoter.

59. The method of claim 57, wherein said promoter is a constitutive promoter.

60. The method of claim 57, wherein said otsA and otsB genes are on two separate expression vectors.

61. The method of claim 60, wherein said two separate expression vectors are present at a ratio of approximately two otsA containing expression vectors to 1 otsB containing expression vector.

62. The mammalian cell of claim 23, wherein otsA and otsB gene functions are on a single gene.

63. The method of claim 53, wherein said cell is stably transfected.

64. The method of claim 53, wherein said cell is a keratinocyte.

65. The method of claim 64, wherein said keratinocyte is a NIKS cell.

66. The method of claim 53, wherein said cells are in an organ.

67. The method of claim 66, wherein said organ is skin.

68. The method of claim 66, wherein said organ is a human skin equivalent.

69. The method of claim 66, wherein said organ comprises NIKS cells.

70. The method of claim 54, wherein said drying comprises freeze drying.

71. The method of claim 54, wherein said drying comprises air or vacuum drying.

72. A method of freezing mammalian cells comprising:
a) providing immortalized keratinocyte cells, wherein said cells contain trehalose and are treated extracellularly with trehalose;
b) treating said cells with an oxyanion;
c) drying said cells.

73. The method of claim 72, further comprising the step of freezing said cells prior to drying said cells.

74. The method of claim 72, wherein said oxyanion is phosphate.

75. A method of treating a patient comprising:
a) providing a patient suffering from a condition and an organ preserved by drying;
b) treating said patient with said organ preserved by drying under conditions such that said condition is relieved.

76. The method of claim 75, wherein said patient is suffering from a condition selected from the group consisting of a burn, wound, donor site wound, and ulcer.

77. The method of claim 75, wherein said organ comprises cells expressing an exogenous trehalose transporter protein.

78. The method of claim 75, wherein said organ comprises cells expressing a trehalose synthesis pathway.

79. A method of preserving mammalian cells comprising
a) providing cells comprising a gene encoding a trehalose synthesis pathway;
b) culturing said cells under conditions such that said cells comprise intracellular trehalose at a concentration of at least 5 mM; and
c) drying said mammalian cells.
